(19) United States
(12) Patent Application Publication
(10) Pub. No.: US 2026/0242437 A1
(43) Pub. Date: Aug. 20, 2026

(54) IL-2 VARIANTS AND USES THEREOF

(71) Applicant: BEIJING MABWORKS BIOTECH CO.,LTD, Beijing (CN)

(72) Inventors: Guangzhong LIN, Beijing (CN); Jiangmei LI, Beijing (CN); Wenqi HU, Beijing (CN); Feng LI, Beijing (CN)

(21) Appl. No.: 19/162,226

(22) PCT Filed: Feb. 6, 2024
(86) PCT No.: PCT/CN2024/076336

§ 371 Date: Sep. 4, 2025

(30) Foreign Application Priority Data
Mar. 9, 2023 (CN) 202310224155.2

(51) Int. Cl. C07K 14/55; A61K 39/00; C07K 16/28

(52) U.S. Cl. CPC C07K 14/55; C07K 16/2803; C07K 16/2818; A61K 2039/505; C07K 2317/14; C07K 2317/31; C07K 2317/92; C07K 2319/00

(57) ABSTRACT

Disclosed is an interleukin-2 (IL-2) variant with decreased binding affinity to the IL-2 receptor, and a recombinant protein containing the IL-2 variant.

This application claims priority to Chinese Patent Application No. 2023102241552 filed on Mar. 9, 2023.

### FIELD OF THE INVENTION

The present disclosure relates to an interleukin-2 (IL-2) variant that has decreased IL-2 receptor binding ability and improved drug-likeness than the wildtype IL-2 molecule. The disclosure also relates to a recombinant protein comprising the IL-2 variant, and its use in treating e.g., cancers and autoimmune diseases.

### BACKGROUND OF THE INVENTION

The immunotherapies have achieved notable clinical efficacy in treatment of many diseases, but the patients' responses to such therapies are not always sustainable. It is important to investigate the underlying mechanism of drug resistance, whether primary or acquired, and figure out a way to overcome this problem. To use the cytokine(s) during the immunotherapy may help, as the cytokine(s) may enhance immune cell infiltration into tumors.

IL-2 is one of the most studied cytokines. It is mainly produced by activated CD4⁺ helper T cells and exerts multiple immunomodulatory effects (J Leukoc Biol. (2018) 103(4):643-655; Sci Immunol. (2018) 3(25):eaat1482). On one side, IL-2 may expand and activate innate and adaptive immune cells (such as T cells and natural killing (NK) cells), initiating immunity. On the other side, IL-2 plays an important role in the maintenance and expansion of immunoinhibitory CD4⁺ CD25⁺ regulatory T cells (Tregs), and may trigger activation induced cell death (AICD) in T cells (Nat Rev Immunol. (2018) 18(10):648-659). IL-2 exerts these functions by binding different IL-2 receptors (IL-2R). There are two types of IL-2 receptor. One is called the intermediate-affinity receptor, consisting of IL2Rβ (CD122) and IL2Rγ (CD132), and expressed on naïve CD4⁺ and CD8⁺ T cells, memory T cells and NK cells. The other type is the high-affinity receptor composed of the IL2Rα, IL2Rβ and IL2Rγ subunits, which is formed with TCR binding or IL-2-induced IL2Rα (CD25) expression, and mainly expressed by Tregs and newly activated effector T cells (Nature. (2012) 484(7395): 529-533). IL-2 binds the latter receptor with 100-fold higher affinity. Among the 3 subunits of IL-2R, the subunit a is not required for the IL-2 signaling.

The IL-2 molecules have a short half-life, and need to be used in a high dose for a sound efficacy. The IL-2 molecules have been approved for clinical treatment of metastatic renal cell cancer and melanoma (dx.doi.org/10.1016/j.it.2015.10.003). However, the high doses bring serious side effects, while lower doses may direct the IL-2 molecules to Tregs, suppressing immune responses. Therefore, the therapeutic window is quite narrow for IL-2, limiting its clinical application (Annu. Rev. Med. (2021) 72:30.1-30.31). Further, the studies have shown pulmonary edema and vascular-leak syndrome caused by the IL-2 therapy may be mediated by the interaction of IL-2 with the IL2Rα on pulmonary endothelial cells (Proc Natl Acad Sci USA (2010) 107(26):11906-11911).

There is a need for an IL-2 molecule that has reduced binding ability to IL2Rα, to attenuate IL-2-induced side effects and Treg-mediated immunosuppression while maintaining its effector immune cell activation ability.

Citation or identification of any document in this application is not an admission that such document is available as prior art to the present invention.

### SUMMARY OF THE INVENTION

The inventors of the application, by observing and analyzing the interface between the IL-2 and the IL2Rα, designed and screened out key amino acid residue mutation(s) that reduce(s) IL-2-IL2Rα binding or interaction. The IL-2 variant comprising the amino acid residue mutation(s), has attenuated or even no IL2Rα binding ability as compared to the wild-type IL-2, eliminating the biased binding to the Tregs.

The inventors of the application further designed and screened out amino acid residue mutation(s) that is/are key to decrease IL-2's binding ability to IL2Rβ and IL2Rγ. The resulting IL-2 variant, in addition to the reduced or even no IL2Rα binding ability, also has reduced IL2Rβγ binding capability, to eliminate activation of NK cells and non-disease specific T cells, resulting in decreased cytokine release and thus the corresponding toxicity, therefore expanding IL-2's therapeutic window (Expert Opin Biol Ther. (2006) 6(12):1323-1331; Nat Biotechnol. (2000) 18(11):1197-1202).

In despite of the reduced biased Treg binding and reduced non-specific immune response-mediated side effects, the IL-2 variant of the disclosure has attenuated ability to activate IL-2 signaling pathways in e.g., effector T cells. Therefore, the inventors of the disclosure linked the IL-2 variant to a molecule targeting the immune cells in the lesion area or specified antigens in diseased cells, such as an antibody specifically binding the PD-1 molecule in the tumor infiltrating CD8⁺ T cells, or the CD4 or FOXP3 molecule on Tregs, to trigger targeted IL-2 signaling activation, leading to more focused activation of the effector T cells, e.g., tumor filtrating CD8+ T cells, in the tumor microenvironment, or the Tregs in the lesion area in an autoimmune disease. A recombinant protein of the disclosure, comprising a PD-1 binding domain and an IL-2 variant, may initiate the IL-2 signaling pathway in PD-1⁺ cells with an activity 10-fold, 100-fold, or even 1000-fold higher than that in PD-1⁻ cells. Such a recombinant protein, in addition to immune cell activation, can also relieve the PD-1-PD-L1-mediated immunosuppression. The recombinant protein above may be further added with a binding domain that specifically binds an exhausted immune cell marker such as LAG-3. The resulting multi-specific recombinant protein may bind well with and re-activate exhausted tumor infiltrating CD8⁺ T cells, elevating the anti-tumor efficacy.

Therefore, in a first aspect, the disclosure may provide an IL-2 variant, which may comprise an amino acid residue deletion and a phenylalanine (Phe, F) to alanine (Ala, A) substitution at the sites corresponding to Position 35 and 42 of SEQ ID NO: 1, respectively; an amino acid residue deletion and a tyrosine (Tyr, Y) to arginine (Arg, R) substitution at the sites corresponding to Position 35 and 45 of SEQ ID NO: 1, respectively; or two amino acid residue deletions at the sites corresponding to Position 34 and 35 of SEQ ID NO: 1, respectively. In certain embodiments, the IL-2 variant may comprise an amino acid residue deletion, a Phe to Ala substitution and a Tyr to Arg substitution at the sites corresponding to Position 35, 42 and 45 of SEQ ID NO: 1.

The IL-2 variant of the disclosure may further comprise one or more amino acid mutations selected from the group consisting of a glutamic acid (Glu, E) to glutamine (Gln, Q) substitution at the site corresponding to Position 15 of SEQ ID NO: 1, an aspartic acid (Asp, D) to leucine (Leu, L) substitution at the site corresponding to Position 20 of SEQ ID NO: 1, a serine (Ser, S) to threonine (Thr, T) substitution at the site corresponding to Position 87 of SEQ ID NO: 1, an asparagine (Asn, N) to aspartic acid (Asp, D) substitution at the site corresponding to Position 88 of SEQ ID NO: 1, and a glutamine (Gln, Q) to threonine (Thr, T) substitution at the site corresponding to Position 126 of SEQ ID NO: 1. In certain embodiments, the IL-2 variant may further comprise a D to L substitution, a N to D substitution and/or a Q to T substitution at the sites corresponding to Position 20, 88 and 126 of SEQ ID NO:1, respectively. In certain embodiments, the IL-2 variant may further comprise a D to L substitution at the site corresponding to Position 20 of SEQ ID NO: 1. In certain embodiments, the IL-2 variant may further comprise a N to D substitution at the site corresponding to Position 88 of SEQ ID NO: 1. In certain embodiments, the IL-2 variant may further comprise a Q to T substitution at the site corresponding to Position 126 of SEQ ID NO:1. In certain embodiments, the IL-2 variant may comprise a D to L substitution, an amino acid residue deletion, a F to A substitution, and a Y to R substitution at the sites corresponding to Position 20, 35, 42 and 45 of SEQ ID NO: 1, respectively. In certain embodiments, the IL-2 variant may comprise an amino acid residue deletion, a F to A substitution, a Y to R substitution and a N to D substitution at the sites corresponding to Position 35, 42, 45 and 88 of SEQ ID NO: 1, respectively. In certain embodiments, the IL-2 variant may comprise an amino acid residue deletion, a F to A substitution, a Y to R substitution and a Q to T substitution at the sites corresponding to Position 35, 42, 45 and 126 of SEQ ID NO: 1, respectively.

The IL-2 variant of the disclosure may further a threonine (Thr, T) to alanine (Ala, A) substitution, a threonine (Thr, T) to glycine (Gly, G) substitution, a threonine (Thr, T) to glutamine (Gln, Q) substitution, a threonine (Thr, T) to glutamic acid (Glu, E) substitution, a threonine (Thr, T) to asparagine (Asn, N) substitution, a threonine (Thr, T) to aspartic acid (Asp, D) substitution, a threonine (Thr, T) to arginine (Arg, R) substitution, a threonine (Thr, T) to lysine (Lys, K) substitution, or a threonine (Thr, T) to proline (Pro, P) substitution at the site corresponding to Position 3 of SEQ ID NO: 1. In certain embodiments, the IL-2 variant may further comprise a T to A substitution at the site corresponding to Position 3 of SEQ ID NO: 1.

The IL-2 variant of the disclosure may further comprise a cysteine (Cys, C) to serine (Ser, S) substitution, or a cysteine (Cys, C) to alanine (Ala, A) substitution at the site corresponding to Position 125 of SEQ ID NO: 1. In certain embodiments, the IL-2 variant may further comprise a C to S substitution at the site corresponding to Position 125 of SEQ ID NO: 1.

The IL-2 variant of the disclosure, in certain embodiments, may comprise a T to A substitution, an E to Q substitution, an amino acid residue deletion, a F to A substitution, a Y to R substitution and a C to S substation at sites corresponding to Position 3, 15, 35, 42, 45 and 125 of SEQ ID NO: 1; a T to A substitution, an amino acid residue deletion, a F to A substitution, a Y to R substitution, an S to T substitution and a C to S substitution at the sites corresponding to Position 3, 35, 42, 45, 87 and 125 of SEQ ID NO: 1; a T to A substitution, an amino acid residue deletion, a F to A substitution, a Y to R substitution, a N to D substitution, and a C to S substitution at the sites corresponding to Position 3, 35, 42, 45, 88 and 125 of SEQ ID NO: 1; a T to A substitution, an amino acid residue deletion, a F to A substitution, a Y to R substitution, a C to S substitution and a Q to T substitution at the sites corresponding to Position 3, 35, 42, 45, 125 and 126 of SEQ ID NO: 1; or a T to A substitution, a D to L substitution, an amino acid residue deletion, a F to A substitution, a Y to R substitution and a C to S substitution at the sites corresponding to Position 3, 20, 35, 42, 45 and 125 of SEQ ID NO: 1. In certain embodiments, the IL-2 variant of the disclosure may comprise a T to A substitution, an amino acid residue deletion, a F to A substitution, a Y to R substitution, a N to D substitution, and a C to S substitution at the sites corresponding to Position 3, 35, 42, 45, 88 and 125 of SEQ ID NO: 1; a T to A substitution, an amino acid residue deletion, a F to A substitution, a Y to R substitution, a C to S substitution, and a Q to T substitution at the sites corresponding to Position 3, 35, 42, 45, 125 and 126 of SEQ ID NO: 1; or a T to A substitution, a D to L substitution, an amino acid residue deletion, a F to A substitution, a Y to R substitution, and a C to S substitution at the sites corresponding to Position 3, 20, 35, 42, 45 and 125 of SEQ ID NO: 1. In certain embodiments, the IL-2 variant of the disclosure may comprise the amino acid sequence of SEQ ID NOs: 5, 6, 7, 16, 17, 18, 19 or 20. In certain embodiments, the IL-2 variant of the disclosure may comprise an amino acid sequence having at least 95%, 96%, 97%, 98% or 99% sequence identity to SEQ ID NOs: 5, 6, 7, 18, 19 or 20. In certain embodiments, the IL-2 variant of the disclosure may comprise the amino acid sequence of SEQ ID NOs: 5, 6, 7, 18, 19 or 20. In certain embodiments, the IL-2 variant of the disclosure may comprise an amino acid sequence having at least 98% or 99% sequence identity to SEQ ID NOs: 18 or 20. In certain embodiments, the IL-2 variant of the disclosure may comprise an amino acid sequence having at least 98% or 99% sequence identity to SEQ ID NOs: 20.

In a second aspect, the disclosure provides a recombinant protein, which may comprise i) an IL-2 variant of the disclosure, ii) an optional linker, and iii) an Fe region of an immunoglobulin heavy chain. In certain embodiments, the recombinant protein of the disclosure may comprise, from the N terminus to the C terminus, i) the IL-2 variant, ii) the optional linker, and iii) the Fc region of the immunoglobulin heavy chain.

The Fc region may be from a human IgG1, IgG2 or IgG4 heavy chain constant region, with or without a binding affinity to an Fe receptor and/or a complement system protein. In certain embodiments, the Fe region may comprise the amino acid sequence of SEQ ID NO: 8.

The linker may be a polypeptide of about 5-30 amino acid residues. In certain embodiments, the linker may be a polypeptide of about 5-20 amino acid residues. In certain embodiments, the linker may be a GS linker, e.g., a GS linker comprising the amino acid sequence of SEQ ID NO: 9.

The recombinant protein of the disclosure, in certain embodiments, may comprise the amino acid sequence of SEQ ID NOs: 10, 11, 12, 13 or 14.

In a third aspect, the disclosure provides a recombinant protein, which may comprise i) an IL-2 variant of the disclosure, and ii) an antigen binding domain targeting a non-IL-2 molecule, wherein the IL-2 variant is linked to the antigen binding domain. The ratio of the IL-2 variant to the antigen binding domain may be 1:1 or 1:2, particularly 1:2, in number.

The antigen binding domain may specifically target an antigen in the lesion site. For example, the antigen binding domain may specifically target a specified area, e.g., a tumor site (e.g., a tumor microenvironment), or an autoimmune disease lesion site, or certain cells in that specified area, e.g., the immune cells or tumor cells in the tumor lesion site, or the Tregs in the autoimmune disease lesion site. The antigen to be targeted in the tumor lesion site may be e.g., carcinoembryonic antigen (CEA), fibroblast activation protein (FAP), PD-1 and the like. The antigen to be targeted in the autoimmune disease lesion site may be e.g., CD4 or FOXP3.

In an embodiment, the recombinant protein may comprise i) an IL-2 variant of the disclosure, and ii) an antigen binding domain targeting PD-1.

The antigen binding domain targeting PD-1 may be an anti-PD-1 antibody or an antigen binding portion thereof. The anti-PD-1 antibody or antigen binding portion thereof may be an IgG antibody, a Fab fragment, a F(ab′)₂ fragment, or the like, as long as it can be linked to the IL-2 variant of the disclosure and maintain its antigen binding ability. The anti-PD-1 antibody or antigen binding portion thereof may comprise a heavy chain variable region and a light chain variable region, wherein the heavy chain variable region may comprise a VH-CDR1, a VH-CDR2, and a VH-CDR3, the light chain variable region may comprise a VL-CDR1, a VL-CDR2 and a VL-CDR3, wherein the VH-CDR1, the VH-CDR2, the VH-CDR3, the VL-CDR1, the VL-CDR2 and the VL-CDR3 may comprise, or consist of, the amino acid sequences of SEQ ID NOs: 21-26, respectively. In certain embodiments, the heavy chain variable region and the light chain variable region may comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 27 and 28, respectively.

The IL-2 variant of the disclosure may be linked to the N terminus or C terminus of the heavy chain or the light chain of the anti-PD-1 antibody or antigen binding portion thereof, as long as the IL-2 variant and the anti-PD-1 antibody or antigen binding portion thereof can exert their respective functions.

The IL-2 variant and the antigen binding domain targeting PD-1 may be in a 1:1 or 1:2 ratio in number. In an embodiments, the IL-2 variant and the antigen binding domain targeting PD-1 may be in a 1:2 ratio.

In an embodiment, the antigen binding domain targeting PD-1 may be an anti-PD-1 full-length antibody, and the N terminus of the IL-2 variant is linked, optionally via a linker, to the C terminus of the heavy chain of the anti-PD-1 full-length antibody.

In certain embodiments, the recombinant protein may comprise:
i) a first polypeptide, comprising an anti-PD-1 heavy chain variable region, a heavy chain constant region, and an IL-2 variant,
ii) a second polypeptide, comprising an anti-PD-1 light chain variable region,
iii) a third polypeptide, comprising an anti-PD-1 heavy chain variable region, and a heavy chain constant region, and
iv) a fourth polypeptide, comprising an anti-PD-1 light chain variable region,
wherein the anti-PD-1 heavy chain variable region in the first polypeptide and the anti-PD-1 light chain variable region in the second polypeptide may form an antigen binding domain targeting PD-1, the anti-PD-1 heavy chain variable region in the third polypeptide and the anti-PD-1 light chain variable region in the fourth polypeptide may form an antigen binding domain targeting PD-1, the heavy chain constant region in the first polypeptide and the heavy chain constant region in the third polypeptide may be associated together via e.g., the knob-into-hole structure, covalent bond(s) or disulfide bond(s).

In certain embodiments, the recombinant protein may comprise:
i) a first polypeptide, comprising, from N terminus to C terminus, an anti-PD-1 heavy chain variable region, a heavy chain constant region, and an IL-2 variant; or an IL-2 variant, an anti-PD-1 heavy chain variable region, and a heavy chain constant region,
ii) a second polypeptide, comprising an anti-PD-1 light chain variable region,
iii) a third polypeptide, comprising, from N terminus to C terminus, an anti-PD-1 heavy chain variable region, and a heavy chain constant region, and
iv) a fourth polypeptide, comprising an anti-PD-1 light chain variable region.

The recombinant protein, in an embodiment, may comprise:
i) a first polypeptide, comprising an anti-PD-1 heavy chain variable region, and a heavy chain constant region,
ii) a second polypeptide, comprising an anti-PD-1 light chain variable region, and an IL-2 variant,
iii) a third polypeptide, comprising an anti-PD-1 heavy chain variable region, and a heavy chain constant region, and
iv) a fourth polypeptide, comprising an anti-PD-1 light chain variable region,
wherein the anti-PD-1 heavy chain variable region in the first polypeptide and the anti-PD-1 light chain variable region in the second polypeptide may form an antigen binding domain targeting PD-1, the anti-PD-1 heavy chain variable region in the third polypeptide and the anti-PD-1 light chain variable region in the fourth polypeptide may form an antigen binding domain targeting PD-1, the heavy chain constant region in the first polypeptide and the heavy chain constant region in the third polypeptide may be associated together.

In certain embodiments, the recombinant protein may comprise:
i) a first polypeptide, comprising, from N terminus to C terminus, an anti-PD-1 heavy chain variable region, and a heavy chain constant region,
ii) a second polypeptide, comprising, from N terminus to C terminus, an IL-2 variant, and an anti-PD-1 light chain variable region,
iii) a third polypeptide, comprising, from N terminus to C terminus, an anti-PD-1 heavy chain variable region, and a heavy chain constant region, and
iv) a fourth polypeptide, comprising an anti-PD-1 light chain variable region.

The second polypeptide and the fourth polypeptide, in an embodiment, may respectively comprise a light chain constant region, e.g., human γ light chain constant region, at the C terminus.

The heavy chain constant regions in the first polypeptide and the third polypeptide, in an embodiment, may be with weak or no FcR binding affinity, particularly with no FcR binding affinity, such as human IgG1 constant region (N297A), human IgG1 constant region (L234A+L235A), human IgG1 constant region (L234A+L235A+P329G/A), human IgG1 constant region (L234A+L235A+N297A), human IgG1 constant region (L234A+L235A+N297A+P329G/A), human IgG2 constant region (V234A+V237A), human IgG1 constant region (L234A+V235E), or human IgG4 constant region.

The heavy chain constant region from one of the first polypeptide and the third polypeptide may be with a knob mutation, e.g., human IgG1 or IgG4 constant region with a T366W mutation, or a functional fragment thereof. The heavy chain constant region from the other of the first polypeptide and the third polypeptide may be with a hole mutation, e.g., human IgG1 or IgG4 constant region with T366S/L368A/Y407V mutations, or a functional fragment thereof.

In certain embodiments, the heavy chain constant region from one of the first polypeptide and the third polypeptide may be with a knob mutation and weak or no FcR binding affinity, e.g., human IgG1 constant region with L234A/L235A/P329A/T366W mutations, or a functional fragment thereof. The heavy chain constant region from the other of the first polypeptide and the third polypeptide may be with a hole mutation and weak or no FcR binding affinity, e.g., human IgG1 constant region with L234A/L235A/P329A/T366S/L368A/Y407V mutations, or a functional fragment thereof.

The IL-2 variant may be linked via a linker to the anti-PD-1 heavy chain variable region, the anti-PD-1 light chain variable region, or the heavy chain constant region. The linker may be a polypeptide of about 5-30 amino acid residues. In certain embodiments, the linker may be a polypeptide of about 5-20 amino acid residues. In certain embodiments, the linker may be a GS linker, e.g., a GS linker comprising the amino acid sequence of SEQ ID NO: 9.

The first, second, third and fourth polypeptides, in certain embodiments, may respectively comprise amino acid sequences having at least 85%, 86%, 87%, 88%, 89%, 90%, 91%, 92%, 93%, 94%, 95%, 96%, 97%, 98%, 99% or 100% sequence identity to i) SEQ ID NOs: 31, 30, 29 and 30; ii) SEQ ID NOs: 32, 30, 29 and 30; iii) SEQ ID NOs: 33, 30, 29 and 30; iv) SEQ ID NOs: 34, 30, 29 and 30; v) SEQ ID NOs: 35, 30, 29 and 30; or vi) SEQ ID NOs: 36, 30, 29 and 30. In certain embodiments, the first, second, third and fourth polypeptides may respectively comprise amino acid sequences having at least 85%, 86%, 87%, 88%, 89%, 90%, 91%, 92%, 93%, 94%, 95%, 96%, 97%, 98%, 99% or 100% sequence identity to i) SEQ ID NOs: 34, 30, 29 and 30; ii) SEQ ID NOs: 35, 30, 29 and 30; or iii) SEQ ID NOs: 36, 30, 29 and 30.

In addition to the lesion site, e.g., the tumor lesion site (the tumor microenvironment) or the autoimmune disease lesion site, the antigen binding domain may also target an antigen expressed on an exhausted immune cell. The antigen expressed on an exhausted immune cell may be e.g., LAG-3, or TIM-3.

The recombinant protein of the disclosure may comprise i) an IL-2 variant of the disclosure, ii) an antigen binding domain targeting an antigen in a lesion site, and iii) an antigen binding domain targeting an antigen expressed on an exhausted immune cell. The three components may be present in a 1:1:1 or 2:1:1 ratio in number. The IL-2 variant of the disclosure may be linked to the antigen binding domain targeting a lesion site, or to the antigen binding domain targeting an antigen expressed on an exhausted immune cell, or to both.

In certain embodiments, the antigen binding domain targeting a lesion site may be an antigen binding domain targeting PD-1, e.g., an anti-PD-1 antibody or an antigen binding portion thereof. The anti-PD-1 antibody or the antigen binding portion thereof may be an IgG antibody, a Fab fragment, a F(ab′)₂ fragment and the like, as long as it can be linked to an IL-2 variant and maintain the PD-1 binding ability.

In certain embodiments, the antigen binding domain targeting an antigen expressed on an exhausted immune cell may be an antigen binding domain targeting LAG-3, e.g., an anti-LAG-3 antibody or an antigen binding portion thereof. The anti-LAG-3 antibody or the antigen binding portion thereof may be an IgG antibody, a Fab fragment, a F(ab′)₂ fragment and the like, as long as it can be linked to an IL-2 variant and maintain the LAG-3 binding ability. The anti-LAG-3 antibody or the antigen binding portion thereof may comprise a heavy chain variable region and a light chain variable region, wherein the heavy chain variable region may comprise a VH-CDR1, a VH-CDR2, and a VH-CDR3, the light chain variable region may comprise a VL-CDR1, a VL-CDR2 and a VL-CDR3, wherein the VH-CDR1, the VH-CDR2, the VH-CDR3, the VL-CDR1, the VL-CDR2 and the VL-CDR3 may comprise, or consist of, the amino acid sequences of SEQ ID NOs: 37-42, respectively. In certain embodiments, the heavy chain variable region and the light chain variable region may comprise amino acid sequences having at least 85%, 86%, 87%, 88%, 89%, 90%, 91%, 92%, 93%, 94%, 95%, 96%, 97%, 98%, 99% or 100% sequence identity to SEQ ID NOs: 43 and 44, respectively.

The IL-2 variant of the disclosure may be linked to the N terminus or C terminus of the heavy chain or the light chain of the anti-PD-1 antibody or antigen binding portion thereof, as long as the IL-2 variant and the anti-PD-1 antibody or antigen binding portion thereof can exert their respective functions. The IL-2 variant of the disclosure may be linked to the N terminus or C terminus of the heavy chain or the light chain of the anti-LAG-3 antibody or antigen binding portion thereof, as long as the IL-2 variant and the anti-LAG-3 antibody or antigen binding portion thereof can exert their respective functions.

The IL-2 variant, the antigen binding domain targeting PD-1 and the antigen binding domain targeting LAG-3 may be present in a 1:1:1 ratio in number. In an embodiment, the IL-2 variant may be linked to the antigen binding domain targeting PD-1 at e.g., the C terminus of the heavy chain.

In certain embodiments, the recombinant protein may comprise:
i) a first polypeptide, comprising an anti-PD-1 heavy chain variable region, a heavy chain constant region, and an IL-2 variant,
ii) a second polypeptide, comprising an anti-PD-1 light chain variable region,
iii) a third polypeptide, comprising an anti-LAG-3 heavy chain variable region, and a heavy chain constant region, and
iv) a fourth polypeptide, comprising an anti-LAG-3 light chain variable region,
wherein the anti-PD-1 heavy chain variable region in the first polypeptide and the anti-PD-1 light chain variable region in the second polypeptide may form an antigen binding domain targeting PD-1, the anti-LAG-3 heavy chain variable region in the third polypeptide and the anti-LAG-3 light chain variable region in the fourth polypeptide may form an antigen binding domain targeting LAG-3, the heavy chain constant region in the first polypeptide and the heavy chain constant region in the third polypeptide may be associated together.

In certain embodiments, the recombinant protein may comprise:
i) a first polypeptide, comprising, from N terminus to C terminus, an anti-PD-1 heavy chain variable region, a heavy chain constant region, and an IL-2 variant; or an IL-2 variant, an anti-PD-1 heavy chain variable region, and a heavy chain constant region,
ii) a second polypeptide, comprising an anti-PD-1 light chain variable region,
iii) a third polypeptide, comprising, from N terminus to C terminus, an anti-LAG-3 heavy chain variable region, and a heavy chain constant region, and
iv) a fourth polypeptide, comprising an anti-LAG-3 light chain variable region.

In certain embodiments, the recombinant protein may comprise:
i) a first polypeptide, comprising an anti-PD-1 heavy chain variable region, and a heavy chain constant region,
ii) a second polypeptide, comprising an anti-PD-1 light chain variable region, and an IL-2 variant,
iii) a third polypeptide, comprising an anti-LAG-3 heavy chain variable region, and a heavy chain constant region, and
iv) a fourth polypeptide, comprising an anti-LAG-3 light chain variable region,
wherein the anti-PD-1 heavy chain variable region in the first polypeptide and the anti-PD-1 light chain variable region in the second polypeptide may form an antigen binding domain targeting PD-1, the anti-LAG-3 heavy chain variable region in the third polypeptide and the anti-LAG-3 light chain variable region in the fourth polypeptide may form an antigen binding domain targeting LAG-3, the heavy chain constant region in the first polypeptide and the heavy chain constant region in the third polypeptide may be associated together.

In certain embodiments, the recombinant protein may comprise:
i) a first polypeptide, comprising, from N terminus to C terminus, an anti-PD-1 heavy chain variable region, and a heavy chain constant region,
ii) a second polypeptide, comprising, from N terminus to C terminus, an IL-2 variant, and an anti-PD-1 light chain variable region,
iii) a third polypeptide, comprising, from N terminus to C terminus, an anti-LAG-3 heavy chain variable region, and a heavy chain constant region, and
iv) a fourth polypeptide, comprising an anti-LAG-3 light chain variable region.

The second polypeptide and the fourth polypeptide, in an embodiment, may respectively comprise a light chain constant region, e.g., human γ light chain constant region, at the C terminus.

The heavy chain constant regions in the first polypeptide and the third polypeptide, in an embodiment, may be with weak or no FcR binding affinity, particularly with no FcR binding affinity, such as human IgG1 constant region (N297A), human IgG1 constant region (L234A+L235A), human IgG1 constant region (L234A+L235A+P329G/A), human IgG1 constant region (L234A+L235A+N297A), human IgG1 constant region (L234A+L235A+N297A+P329G/A), human IgG2 constant region (V234A+V237A), human IgG1 constant region (L234A+V235E), or human IgG4 constant region.

The heavy chain constant region from one of the first polypeptide and the third polypeptide may be with a knob mutation, e.g., human IgG1 or IgG4 constant region with a T366W mutation, or a functional fragment thereof. The heavy chain constant region from the other of the first polypeptide and the third polypeptide may be with a hole mutation, e.g., human IgG1 or IgG4 constant region with T366S/L368A/Y407V mutations, or a functional fragment thereof.

In certain embodiments, the heavy chain constant region from one of the first polypeptide and the third polypeptide may be with a knob mutation and weak or no FcR binding affinity, e.g., human IgG1 constant region with L234A/L235A/P329A/T366W mutations or a functional fragment thereof. The heavy chain constant region from the other of the first polypeptide and the third polypeptide may be with a hole mutation and weak or no FcR binding affinity, e.g., human IgG1 constant region with L234A/L235A/P329A/T366S/L368A/Y407V mutations, or a functional fragment thereof.

The IL-2 variant may be linked via a linker to the anti-PD-1 or anti-LAG-3 heavy chain variable region, the anti-PD-1 or anti-LAG-3 light chain variable region, or the heavy chain constant region. The linker may be a polypeptide of about 5-30 amino acid residues. In certain embodiments, the linker may be a polypeptide of about 5-20 amino acid residues. In certain embodiments, the linker may be a GS linker, e.g., a GS linker comprising the amino acid sequence of SEQ ID NO: 9.

The first, second, third and fourth polypeptides, in certain embodiments, may respectively comprise amino acid sequences having at least 85%, 86%, 87%, 88%, 89%, 90%, 91%, 92%, 93%, 94%, 95%, 96%, 97%, 98%, 99% or 100% sequence identity to i) SEQ ID NOs: 31, 30, 45 and 46; ii) SEQ ID NOs: 32, 30, 45 and 46; iii) SEQ ID NOs: 33, 30, 45 and 46; iv) SEQ ID NOs: 34, 30, 45 and 46; v) SEQ ID NOs: 35, 30, 45 and 46; or vi) SEQ ID NOs: 36, 30, 45 and 46. In certain embodiments, the first, second, third and fourth polypeptides may respectively comprise amino acid sequences having at least 85%, 86%, 87% 88%, 89%, 90%, 91%, 92%, 93%, 94%, 95%, 96%, 97%, 98%, 99% or 100% sequence identity to i) SEQ ID NOs: 31, 30, 45 and 46; ii) SEQ ID NOs: 34, 30, 45 and 46; iii) SEQ ID NOs: 35, 30, 45 and 46; or iv) SEQ ID NOs: 36, 30, 45 and 46.

In a fourth aspect, the disclosure provides a nucleic acid molecule that may encode the IL-2 variant or the recombinant protein of the disclosure, an expression vector that may comprise the nucleic acid molecule, and a host cell that may comprise the expression vector or have the nucleic acid molecule integrated into its genome. The disclosure also provides a method for preparing an IL-2 variant or a recombinant protein of the disclosure using the host cell, comprising i) allowing the host cell to express the IL-2 variant or the recombinant protein, and ii) isolating the IL-2 variant or the recombinant protein from the host cell or its culture.

The disclosure further provides a composition which may comprise the IL-2 variant, the recombinant protein, the nucleic acid molecule, the expression vector, or the host cell, of the disclosure. In certain embodiments, the composition may be a pharmaceutical composition comprising a therapeutically effective amount of the IL-2 variant, the recombinant protein, the nucleic acid molecule, the expression vector, or the host cell, and a pharmaceutically acceptable carrier.

In a fifth aspect, the disclosure provides a method for treating or alleviating tumor, or activating an immune cell in a tumor microenvironment in a subject in need thereof, comprising administering to the subject the pharmaceutical composition of the disclosure. The pharmaceutical composition may comprise an IL-2 variant, a recombinant protein, or a nucleic acid molecule, an expression vector or a host cell for preparing the same, of the disclosure. The recombinant protein may comprise an IL-2 variant and an antigen binding domain targeting a specified antigen in the tumor lesion site. The specified antigen in the tumor lesion site may be an antigen expressed by a certain cell such as a tumor cell or an immune cell, e.g., carcinoembryonic antigen (CEA), fibroblast activation protein (FAP), PD-1 and the like. Alternatively, the recombinant protein may comprise an IL-2 variant, an antigen binding domain targeting a specified antigen in the tumor lesion site, and an antigen binding domain targeting an antigen expressed by an exhausted immune cell. The antigen expressed by an exhausted immune cell may be e.g., LAG-3 or TIM-3. The tumor to be treated in the disclosure may be a solid tumor or a hematological tumor, including but not limited to colon cancer and pancreatic cancer.

The disclosure also provides a method for treating or alleviating an autoimmune disease in a subject in need thereof, comprising administering to the subject the pharmaceutical composition of the disclosure. The pharmaceutical composition may comprise a recombinant protein, or a nucleic acid molecule, an expression vector or the host for preparing the same, of the disclosure. The recombinant protein may comprise an IL-2 variant and an antigen binding domain targeting a Treg in the autoimmune disease lesion site. The antigen associated with a Treg may be e.g., CD4 or FOXP3.

In certain embodiments, the subject may be a mammal, especially human.

The disclosure provides the use of the IL-2 variant, the recombinant protein, the nucleic acid molecule, the expression vector, the host cell or the pharmaceutical composition of the disclosure in preparation of a medicament for treating or alleviating a tumor or an autoimmune disease, or activating an immune cell in a tumor lesion site.

The disclosure also provides a method for in vitro activating an immune cell, comprising contacting the immune cell with the IL-2 variant, the recombinant protein, the nucleic acid molecule, the expression vector or the host cell of the disclosure. The immune cell may be a naïve immune cell, or an exhausted immune cell, such as an effector T cell, particularly an exhausted effector T cell.

Other features and advantages of the instant disclosure will be apparent from the following detailed description and examples, which should not be construed as limiting. The contents of all references, Genbank entries, patents and published patent applications cited throughout this application are expressly incorporated herein by reference.

### BRIEF DESCRIPTION OF THE DRAWINGS

The following detailed description, given by way of example, but not intended to limit the invention solely to the specific embodiments as described, may best be understood in conjunction with the accompanying drawings.

FIG. 1 is a schematic diagram showing the interaction between an IL-2 molecule and a CD25 molecule located above and below the dashed line respectively (A), and a merge of a free IL-2 molecule (in dark grey) and a CD25 bound IL-2 molecule (in light grey) (B) with significant conformational change occurring at amino acid residues 31-35 located in the dashed circle.

FIG. 2 is a schematic diagram of the IL-2-containing recombinant proteins, i.e., a Fc-IL-2 fusion protein (A), a bispecific recombinant protein containing a PD-1 binding domain and an IL-2 (B) and a tri-specific recombinant protein containing a PD-1 binding domain, a LAG-3 binding domain and an IL-2 (C).

FIG. 3 shows the binding activity of the Fc-IL-2 furoin protein containing a wildtype IL-2 or an IL-2 variant to the CD25 molecule.

FIG. 4 shows the binding activity of the Fc-IL-2 furoin protein containing a wildtype IL-2 or an IL-2 variant to Tregs (A) or effector T cells (B).

FIG. 5 shows the activity of the Fc-IL-2 fusion protein containing a wildtype IL-2 or an IL-2 variant to trigger the signaling pathways in HEK blue/IL-2 cells.

FIG. 6 is a schematic diagram showing the interaction of an IL-2 molecule (below the dashed line) with the IL-2Rβ (above the dashed line, left) or IL-2Rγ (above the dashed line, right).

FIG. 7 shows the activity of the bispecific recombinant protein containing an IL-2 variant and a PD-1 binding domain to initiate the signaling pathways in PD-1⁻HEK blue-IL-2 cells (A) or PD-1 HEK blue/IL-2 cells (B).

FIG. 8 shows the binding capability of the bispecific recombinant protein containing an IL-2 variant and a PD-1 binding domain to HEK293A/PD-1 cells.

FIG. 9 shows the activity of the bispecific recombinant protein containing an IL-2 variant and a PD-1 binding domain to induce CD3⁺ T cell proliferation (A) and activation (B).

FIG. 10 shows the average tumor size (A) and average body weight (B) of tumor-bearing mice treated with the bispecific recombinant protein containing an IL-2 variant and a PD-1 binding domain.

FIG. 11 shows the average tumor size (A), average body weight (B) and overall survival (C) of tumor-bearing mice treated with the bispecific recombinant protein.

FIG. 12 shows the plasma concentration of the bispecific recombinant protein in mice.

FIG. 13 shows the binding capability of a bispecific recombinant protein containing an IL-2 variant and a PD-1 binding domain, and a tri-specific recombinant protein containing a PD-1 binding domain, a LAG-3 binding domain and an IL-2 variant to isolated primary naïve CD3⁺ T cells (A) or exhausted CD3⁺ T cells (B).

### DETAILED DESCRIPTION OF THE INVENTION

To ensure that the present disclosure may be more readily understood, certain terms are first defined. Additional definitions are set forth throughout the detailed description.

The term “interleukin-2” or “IL-2” refers to the IL-2 molecule of the vertebrate, including the mammals such as the primates (e.g., human). This term may include the naturally occurring IL-2 molecules and the genetically engineered IL-2 molecules, wild-type or with modifications. Exemplary IL-2 molecules may comprise the amino acid sequence of SEQ ID NOs: 1, 2 or 3.

The term “IL-2 variant” refers to an IL-2 mutant, including the full-length IL-2, and the truncated IL-2. Compared to a wild-type IL-2, an IL-2 variant may contain at least one amino acid mutation that affects its interaction with any one of the IL-2 receptor subunits. The amino acid mutation may be an amino acid residue substitution, an amino acid residue deletion, an amino acid residue addition, or an amino acid residue modification. The mutation(s) in the IL-2 variant may occur at the site(s) corresponding to the position(s) of SEQ ID NO: 1. For example, 42A, F42A or Phe42Ala may mean that the phenylalanine in the IL-2 molecule at the site corresponding to Position 42 of SEQ ID NO: 1 is replaced with alanine. An IL-2 variant may differ from a wild-type IL-2 molecule only in the specified amino acid mutation(s). For example, the wild-type IL-2 molecule for a full-length IL-2 variant may be of full-length and differ from the full-length IL-2 variant only in the mentioned amino acid mutation(s). The “wild-type” or “wildtype” IL-2 molecule may, compared to the naturally occurring IL-2 molecules, contain one or more amino acid mutations that does/do not affect its binding affinity to the IL-2 receptor. For example, the wild-type IL-2 may contain a T to A, G, Q, E, N, D, R, K or P substitution at the site corresponding to Position 3 of SEQ ID NO: 1, to reduce the occurrence of O-glycosylation, and/or contain a C to S or A substitution at the site corresponding to Position 125 of SEQ ID NO: 1, to reduce the number of disulfide bonds, both aiming to reduce the formation of aggregates. In certain embodiments, the wild-type IL-2 molecule to be compared with the IL-2 variant may comprise the amino acid sequence of SEQ ID NOs: 1, 2 or 3.

The term “amino acid mutation” or “amino acid residue mutation” includes the amino acid substitution, deletion, addition and modification. The IL-2 variant may be obtained through any combination of the substitution, deletion, addition and modification, as long as the construct has the desired characteristics, e.g., reduced binding affinity to IL2Rα, IL2Rβ, IL2Rγ or any of their combination. The amino acid deletion and addition may include the amino acid deletion and addition at the N terminus and/or the C terminus. Particularly, the amino acid mutation may include the deletion, to impair the interaction between the IL-2 and the IL-2 receptor subunit(s). Particularly, the amino acid mutation may be amino acid substitution. To modify e.g., the binding property of the IL-2 molecule, non-conservative amino acid substitution may be done, i.e., one amino acid may be replaced by another having a different structure and/or different chemical properties. Particularly, a hydrophilic amino acid can be replaced with a hydrophobic amino acid. The amino acid substitution can be done with naturally occurring amino acids or derivatives of the 20 naturally occurring amino acids (e.g., 4-hydroxyproline, 3-methylhistone, ornithine, homoserine, 5-hydroxylysine). The amino acid mutation can be performed with the well-known genetic or chemical methods in the art, including, but not limited to, site-directed mutagenesis, PCR and gene synthesis. The disclosure also covers chemical modifications that bring changes to the side group(s) of the amino acid(s).

The term “IL-2R alpha subunit”, “IL-2Rα” or “CD25” refers to the CD25 molecule of the vertebrate, including the mammal such as the primate (e.g., human). This term may include the naturally occurring CD25 molecules, unprocessed and full-length, or processed, including the naturally occurring CD25 variants such as the splice variants. In certain embodiments, the CD25 protein may be the human CD25 protein.

The term “IL-2R beta subunit”, “IL-2Rβ” or “CD122” refers to the CD122 molecule of the vertebrate, including the mammal such as the primate (e.g., human). This term may include the naturally occurring CD122 molecules, unprocessed and full-length, or processed, including the naturally occurring CD122 variants such as the splice variants. In certain embodiments, the CD122 protein may be the human CD122 protein.

The term “IL-2R gamma subunit”, “IL-2Rγ” or “CD132” refers to the CD132 molecule of the vertebrate, including the mammal such as the primate (e.g., human). This term may include the naturally occurring CD132 molecules, unprocessed and full-length, or processed, including the naturally occurring CD132 variants such as the splice variants. In certain embodiments, the CD132 protein may be the human CD132 protein.

The term “high-affinity IL-2 receptor” refers to the trimeric receptor consisting of the alpha subunit, beta subunit and the gamma subunit, while the term “intermediate-affinity IL-2 receptor” refers to the dimeric receptor consisting of the beta subunit and the gamma subunit.

The term “effector cell” herein refers to the relatively short-lived activated cells in the immune system that defend the body in an immune response, whose growth, proliferation and differentiation may be mediated by the IL-2 proteins. The effector cell may include effector T cells, such as CD8⁺ regulatory T cells, NK cells, lymphokine-activated killer (LAK) cells and macrophages/monocytes.

The term “regulatory T cell” or “Treg” herein refers to a specialized CD4⁺ T cell subpopulation that may suppress other T cells' immune responses. The Tregs are characterized by expression of the IL-2R alpha subunit (CD25) and forkhead box P3 (FOXP3), and play important roles in inducing and maintaining peripheral tolerance to antigens (including the antigens expressed by tumor cells). The function, development of and the inhibitory characteristics of Tregs are induced or mediated by the IL-2 molecules.

The term “polypeptide” refers to a linear molecule consisting of amino acid residues bonded together via peptide bonds (an amide type of covalent chemical bond) in a chain, including any peptide chain having two or more amino acid residues. Thus, the term may comprise the peptides, dipeptides, tripeptides, oligopeptides, proteins, amino acid chains or any chains containing two or more amino acid residues. The “polypeptide” may further include the polypeptide products with post-translational modifications, including, but not limited to, glycosylation, acetylation, phosphorylation, acylation, modifications via derivation of protective or blocking group(s), modifications via proteolysis, and addition of non-naturally occurring amino acids. The polypeptide may be derived from the natural polypeptides, prepared by genetically engineering means, or chemically synthesized. The polypeptide may be with a specified 3D structure, although it is not necessary.

The term “immunoglobulin” may refer to a naturally occurring protein having the antibody structure.

The term “antibody” as referred to herein includes IgG, IgA, IgD, IgE and IgM whole antibodies and any antigen binding fragment (i.e., “antigen-binding portion”) or single chains thereof. Whole antibodies are glycoproteins comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. The heavy chain constant region of certain antibodies of the disclosure may be designed with weak or no binding affinity to the immune cells or the complement system protein.

The term “antigen-binding portion” of an antibody (or simply “antibody portion”), as used herein, refers to one or more fragments of an antibody that retain the ability to specifically bind to an antigen (e.g., a PD-1 or LAG-3 protein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L} V_{H}, C_{L} and C_{H} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H}H domain; (vi) an isolated complementarity determining region (CDR); and (vii) a single chain variable fragment (scFv) containing a V_{H} and a V_{L} linked via a linker (see e.g., Bird et al., (1988) Science 242:423-426; and Huston et al., (1988) Proc. Natl. Acad. Sci. USA 85:5879-5883). These antibody fragments are obtained using conventional techniques known to those with skill in the art, and the fragments are screened for utility in the same manner as are intact antibodies.

A “bispecific” molecule or recombinant protein herein may refer a molecule that specifically binds to two target molecules or two epitopes in a single target molecule, such as a bispecific antibody. The bispecific recombinant protein includes the one containing a PD-1 binding domain and an IL-2 variant. A “tri-specific” molecule or recombinant protein may refer to a molecule that specifically binds two or more (e.g., three) target molecules or two or more (e.g., three) epitopes on one target molecule, such as a tri-specific antibody. The tri-specific molecule or recombinant protein may include the one containing a PD-1 binding domain, a LAG-3 binding domain, and an IL-2 variant.

As used herein, an antibody that “specifically binds” e.g., human PD-1 is intended to refer to a molecule such as an antibody or an antigen binding portion thereof that can distinguish a target molecule from one or more reference molecules, and binds the target molecule with an affinity that is e.g., 1-fold, 5-fold, or 10-fold higher than that to other reference molecules. The binding specificity can be determined by e.g., Western blot, ELISA, RIA, ECL, IRMA or peptide scanning.

The percent “sequence identity” as used herein in the context of two or more nucleic acids or polypeptides, refers to two or more sequences or subsequences that are the same or have a specified percentage of nucleotides or amino acid residues that are the same, when compared and aligned (introducing gaps, if necessary) for maximum correspondence, considering or not considering conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof.

The term “half antibody” or “half-antibody” refers to one half of an antibody which comprises e.g., a heavy chain and a light chain.

The “knob variant” of a heavy chain constant region, or a heavy chain constant region with “knob mutation(s)” refers to a heavy chain constant region used in the knobs-into-holes technology whose C_{H3} domain is engineered to create a “knob”. Similarly, the “hole variant” of a heavy chain constant region, or a heavy chain constant region with “hole mutation(s)” refers to a heavy chain constant region used in the knobs-into-holes technology whose C_{H3} domain is engineered to create a “hole”.

The term “EC₅₀”, also known as half maximal effective concentration, refers to the concentration of a molecule which induces a response halfway between the baseline and maximum after a specified exposure time.

The term “IC₅₀”, also known as half maximal inhibitory concentration, refers to the concentration of a molecule which inhibits a specific biological or biochemical function by 50% relative to the absence of the molecule.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “therapeutically effective amount” means an amount of a molecule of the present disclosure sufficient to prevent or ameliorate the symptoms associated with a disease or condition (such as a cancer) and/or lessen the severity of the disease or condition. A therapeutically effective amount is understood to be in context to the condition being treated, where the actual effective amount is readily discerned by those of skill in the art.

Various aspects of the disclosure are described in further detail in the following subsections.

### IL-2 Variants

The application aims to provide an IL-2 variant with improved characteristics in immune-therapies.

As mentioned above, there are two kinds of IL-2 receptor, which are consisted of different subunits and show different IL-2 binding affinity. The intermediate-affinity IL-2 receptor consisting of the β and γ subunits is expressed on resting effector cells and sufficient for IL-2 signal transduction. The high-affinity IL-2 receptor further containing the a subunit is mainly found on regulatory T cells and newly activated effector cells, and, when bound by the IL-2 molecules, promotes Treg-mediated immune suppression and activation-induced cell death (AICD) of T cells. Thus, the inventors of the application tried to decrease or eliminate the binding affinity of the IL-2 molecule to the IL-2 receptor a subunit, so as to reduce the IL-2 mediated Treg associated immune suppression and T cell AICD.

Therefore, in one aspect, the disclosure provides an IL-2 variant having reduced or eliminated binding affinity to the IL-2Rα and thus reduced or eliminated biased binding to Tregs compared to the wild-type IL-2. In particular, the IL-2 variant may comprise an amino acid residue deletion and a F to A substitution at the sites corresponding to Position 35 and 42 of SEQ ID NO: 1, respectively; or an amino acid residue deletion and a Y to R substitution at the sites corresponding to Position 35 and 45 of SEQ ID NO: 1, respectively. In certain embodiments, the IL-2 variant of the disclosure may comprise an amino acid residue deletion, a F to A substitution, and a Y to R substitution at the sites corresponding to Position 35, 42 and 45 of SEQ ID NO: 1.

In certain embodiments, the amino acid residue mutation(s) may render the IL-2 variant have at least 5-fold, at least 10-fold, particularly at least 25-fold lower binding affinity to the IL-2Rα subunit. In embodiments where more than one amino acid mutation that reduce the IL-2's binding affinity to IL-2Rα occur, the IL-2 variants may have at least 30-fold, at least 50-fold, particularly at least 100-fold lower binding affinity to the IL-2Rα subunit. In an embodiment, the amino acid mutation(s) may eliminate the IL-2's binding affinity to the IL-2Rα subunit. The binding affinity can be measured by e.g., surface plasmon resonance.

In certain embodiments, the IL-2 variant may induce one or more cell responses selected from the group consisting of proliferation of activated T lymphocytes, differentiation of activated T lymphocytes, activation of cytotoxic T lymphocytes (CTLs), proliferation of activated B cells, differentiation of activated B cells, proliferation of NK cells, differentiation of NK cells, cytokine secretion by activated T cells or NK cells, and lymphokine-activated killer (LAK) cells' cytotoxicity to tumor cells.

In an embodiment, the IL-2 variant may have reduced ability to induce IL-2 signaling in Tregs than the wild-type IL-2. In one embodiment, the IL-2 variant may induce less AICD of T cells than the wild-type IL-2.

The inventors of the disclosure have found that, in addition to the IL2Rα subunit, the IL-2 molecule's binding affinity to the IL-2 receptor β and γ subunits may be reduced to some extent, to decrease its activity to activate NK cells and non-disease specific T cells, such that the adverse side effects may be attenuated.

Thus, in another aspect, the present application provides an IL-2 variant having reduced or eliminated binding affinity to the IL-2Rα and IL-2Rβγ and thus reduced or eliminated biased binding to Tregs as well as reduced or eliminated side effects due to non-specific immune responses, compared to the wild-type IL-2. Particularly, the IL-2 variant may comprise i) an amino acid residue deletion and a F to A substitution at the sites corresponding to Position 35 and 42 of SEQ ID NO: 1; an amino acid residue deletion and a Y to R substitution at the sites corresponding to Position 35 and 45 of SEQ ID NO: 1; or an amino acid residue deletion, a F to A substitution, and a Y to R substitution at the sites corresponding to Position 35, 42 and 45 of SEQ ID NO: 1, and ii) one or more amino acid mutations selected from the group consisting of a E to Q substitution at the site corresponding to Position 15 of SEQ ID NO: 1, a D to L substitution at the site corresponding to Position 20 of SEQ ID NO: 1, a S to T substitution at the site corresponding to Position 87 of SEQ ID NO: 1, a N to D substitution at the site corresponding to Position 88 of SEQ ID NO: 1, and a Q to T substitution at the site corresponding to Position 126 of SEQ ID NO: 1. In certain embodiments, the IL-2 variant may comprise i) an amino acid residue deletion and a F to A substitution at the sites corresponding to Position 35 and 42 of SEQ ID NO: 1; an amino acid residue deletion and a Y to R substitution at the sites corresponding to Position 35 and 45 of SEQ ID NO: 1; or an amino acid residue deletion, a F to A substitution, and a Y to R substitution at the sites corresponding to Position 35, 42 and 45 of SEQ ID NO: 1, and ii) one or more amino acid mutations selected from the group consisting of a D to L substitution at the site corresponding to Position 20 of SEQ ID NO: 1, a N to D substitution at the site corresponding to Position 88 of SEQ ID NO: 1, and a Q to T substitution at the site corresponding to Position 126 of SEQ ID NO: 1. In certain embodiments, the IL-2 variant may comprise a D to L substitution, an amino acid residue deletion, a F to A substitution, and a Y to R substitution at the sites corresponding to Position 20, 35, 42 and 45 of SEQ ID NO: 1. In certain embodiments, the IL-2 variant may comprise an amino acid residue deletion, a F to A substitution, a Y to R substitution, and a N to D substitution at the sites corresponding to Position 35, 42, 45 and 88 of SEQ ID NO: 1. In certain embodiments, the IL-2 variant may comprise an amino acid residue deletion, a F to A substitution, a Y to R substitution, and a Q to T substitution at the sites corresponding to Position 35, 42, 45 and 126 of SEQ ID NO: 1.

The IL-2 variant may comprise, in addition to the mutation(s) at the interface of the IL-2 and each IL-2 receptor subunit, one or more amino acid mutations on other areas. The additional mutation(s) may e.g., facilitate IL-2 expression or stability. For example, the cysteine (Cys, C) at Position 125 of SEQ ID NO: 1 may be replaced by a neutral amino acid such as serine (Ser, S), alanine (Ala, A), threonine (Thr, T), or valine (Val, V), to reduce the count of disulfide bonds in the IL-2, resulting in less aggregate formation (see U.S. Pat. No. 4,518,584). The alanine (Ala, A) at the N terminus of the IL-2 may be deleted, to obtain IL-2 variants with e.g., delA1 C125S or delA1 C125A. Alternatively, the IL-2 variant may have the methionine (Met, M) at Position 104 of the wild-type IL-2 replaced with a neutral amino acid such as alanine (Ala, A) (see U.S. Pat. No. 5,206,344), and the resultant IL-2 with delA1 M104A, delA1 M104A C125S, M104A, M104A C125A, delA1 M104A C125A, or M104A C125S (see U.S. Pat. No. 5,116,943; Weiger et al, (1989) Eur J biochem 180:295-300) may be used in combination with the IL-2 variant(s) of the disclosure.

The IL-2 variant may comprise a T to A, G, Q, E, N, D, R, K or P substitution at the site corresponding to Position 3 of SEQ ID NO: 1. Such an amino acid mutation may remove the O-glycosylation site in the IL-2, resulting in fewer aggregates. In certain embodiments, the IL-2 variant may further comprise a T to A substitution at the site corresponding to Position 3 of SEQ ID NO: 1.

### IL-2 Variant-Containing Recombinant Proteins

The IL-2 variant of the disclosure may be fused to an Fe fragment, to increase the serum half-life.

The IL-2 variant of the disclosure has eliminated biased binding to Tregs, reduced side effects caused by non-specific immune responses, and also reduced ability to activate IL-2 signaling in e.g., effector T cells.

Therefore, to enhance the IL-2 variant's on-target activation ability, targeted IL-2 delivery may be performed. For example, the IL-2 molecules may be delivered to the tumor lesion site to activate effector cells such as tumor filtrating CD8⁺ T cells, or to the immune disease lesion site to activate the Tregs. The inventors of the application achieved the goal by linking the IL-2 variant of the disclosure to an antigen binding domain targeting an immune cell or a diseased cell in the lesion site, e.g., a PD-1 binding domain that targets the PD-1s on tumor filtrating CD8⁺ T cells in tumor therapies, or to an antigen binding domain that targets the CD4 and/or FOXP3 on Tregs in the lesion site in the immune disease treatment. As shown in the Examples part of the disclosure, the recombinant protein containing a PD-1 binding domain and an IL-2 variant activated the IL-2 signaling pathways in PD-1 cells with an activity that was 10-fold, 100-fold, or even 1000-fold higher than that in PD-1⁻ cells. The recombinant protein of the disclosure may further release the PD-1-PD-L1 mediated immune suppression, which may work together with the immune system activation mentioned above.

In certain embodiments, the recombinant protein of the disclosure may comprise i) the IL-2 variant of the disclosure, and ii) an antigen binding domain capable of specifically binding to a non-IL-2 molecule, wherein the IL-2 variant is linked to the antigen binding domain. The antigen binding domain may specifically target an antigen in the lesion site. For example, the antigen binding domain may specifically target a specified area, e.g., a tumor lesion site (e.g., a tumor microenvironment), or an autoimmune disease lesion site, or certain cells in that specified area, e.g., the immune cells or tumor cells in the tumor lesion site, or the Tregs in the autoimmune disease lesion site. The antigen to be targeted in the tumor lesion site may be e.g., carcinoembryonic antigen (CEA), fibroblast activation protein (FAP), PD-1 and the like. The antigen to be targeted in the autoimmune disease lesion site may be e.g., CD4 or FOXP3.

In an embodiment, the recombinant protein may comprise i) the IL-2 variant of the disclosure, and ii) an antigen binding domain targeting PD-1. The IL-2 variant and the PD-1 binding domain may be present in a 1:1 or 1:2 ratio. In one embodiment, the number ratio of the IL-2 variant to the PD-1 binding domain may be 1:2. The PD-1 binding domain may be an anti-PD-1 antibody or its antigen binding portion thereof. The anti-PD-1 antibody or the antigen binding portion thereof may be e.g., an IgG antibody, a Fab fragment, or a F(ab′)₂ fragment, as long as it can be linked to the IL-2 variant of the disclosure and have the antigen binding ability. The IL-2 variant of the disclosure may be linked to an anti-PD-1 antibody or its antigen binding portion thereof at the N terminus of the heavy or light chain, or the C terminus of the heavy chain, as long as the IL-2 variant and the anti-PD-1 antibody or antigen binding portion thereof can exert their respective functions.

In addition to certain lesion sites, e.g., the tumor lesion site (the tumor microenvironment) or the autoimmune disease lesion site, the antigen binding domain may target an antigen expressed on an exhausted immune cell, such that the IL-2 may specifically activate the exhausted immune cell.

Particularly, when the recombinant protein containing the IL-2 variant targets the lesion site and the exhausted immune cell at the same time, the IL-2 molecule may re-activate the exhausted tumor filtrating CD8⁺ T cells, so as to further improve the anti-tumor efficacy. The antigen expressed on an exhausted immune cell may be e.g., LAG-3, or TIM-3.

In certain embodiments, the recombinant protein of the disclosure may comprise i) an IL-2 variant of the disclosure, ii) an antigen binding domain targeting an antigen in a lesion site, and iii) an antigen binding domain targeting an antigen expressed on an exhausted immune cell. The three components may be present in a 1:1:1 or 2:1:1 ratio. The IL-2 variant of the disclosure may be linked to the antigen binding domain targeting a lesion site, or to the antigen binding domain targeting an antigen expressed on an exhausted immune cell, or to both.

In certain embodiments, the antigen binding domain targeting a lesion site may be a PD-1 binding domain, e.g., an anti-PD-1 antibody or an antigen binding portion thereof. The anti-PD-1 antibody or the antigen binding portion thereof may be an IgG antibody, a Fab, a F(ab′)₂ and etc., as long as it can be linked to the IL-2 variant and have PD-1 binding ability. In certain embodiments, the antigen binding domain targeting an antigen expressed on an exhausted immune cell may be a LAG-3 binding domain, e.g., an anti-LAG-3 antibody or an antigen binding portion thereof. The anti-LAG-3 antibody or the antigen binding portion thereof may be an IgG antibody, a Fab, a F(ab′)₂ and etc., as long as it can be linked to the IL-2 variant and have LAG-3 binding ability.

The IL-2 variant of the disclosure may be linked to the N terminus or C terminus of the heavy chain or the light chain variable region, or the C terminus of the heavy chain constant region of the anti-PD-1 antibody or antigen binding portion thereof, as long as the IL-2 variant and the anti-PD-1 antibody or antigen binding portion thereof can exert their respective functions. The IL-2 variant of the disclosure may be linked to the N terminus or C terminus of the heavy chain or the light chain variable region, or the C terminus of the heavy chain constant region of the anti-LAG-3 antibody or antigen binding portion thereof, as long as the IL-2 variant and the anti-LAG-3 antibody or antigen binding portion thereof can exert their respective functions.

The IL-2 variant, the PD-1 binding domain and the LAG-3 binding domain may be present in a 1:1:1 ratio in number. In an embodiment, the IL-2 variant may be linked to the PD-1 binding domain.

### Antibodies or Antigen Binding Portions Thereof

The heavy chain variable region CDRs and light chain variable region CDRs of the antibody or antigen binding portion thereof in the present application used as the non-IL-2 binding domain, have been defined by the Kabat numbering system. However, as is well known in the art, CDRs can also be determined by other systems such as Chothia, and IMGT, AbM, or Contact numbering system/method, based on heavy chain/light chain variable region sequences.

In the construction of the bispecific recombinant protein or the multi- or tri-specific recombinant protein of the disclosure, the IL-2 may be linked to the non-IL-2 binding antibody or antigen binding portion thereof via a linker.

The linker may be made up of amino acids linked together by peptide bonds, preferably from 5 to 30 amino acids linked by peptide bonds, wherein the amino acids are selected from the 20 naturally occurring amino acids. One or more of these amino acids may be glycosylated, as is understood by those of skill in the art. In one embodiment, the 5 to 30 amino acids may be selected from glycine, alanine, proline, asparagine, glutamine, serine and lysine. In one embodiment, a linker is made up of a majority of amino acids that are sterically unhindered, such as glycine and alanine. Exemplary linkers are polyglycines, particularly poly(Gly-Ala), and polyalanines.

The linker may also be a non-peptide linker. For example, alkyl linkers such as —NH—, —(CH₂)₈—C(O)—, wherein s=2-20 can be used. These alkyl linkers may further be substituted by any non-sterically hindering group such as lower alkyl (e.g., C₁₋₄) lower acyl, halogen (e.g., Cl, Br), CN, NH₂, phenyl, etc.

In the construction of the bispecific recombinant protein or the multi- or tri-specific recombinant protein of the disclosure, the heavy chain constant regions of two half-antibodies may be designed with a knob-into-hole structure, to facilitate the assembly of the molecules. That is, one heavy chain constant region may be with a knob mutation, e.g., human IgG1 or IgG4 constant region with a T366W mutation or a functional fragment thereof. The other heavy chain constant region may be with a hole mutation, e.g., human IgG1 or IgG4 constant region with T366S/L368A/Y407V mutations or a functional fragment thereof.

The bi-/multi-specific recombinant protein of the disclosure may comprise a heavy and/or light chain variable region sequences or CDR1, CDR2 and CDR3 sequences with one or more conservative modifications. It is understood in the art that certain conservative sequence modification can be made which do not remove antigen binding. As used herein, the term “conservative sequence modification” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art.

The antibody or antigen binding portion thereof in the bi-/multi-specific recombinant protein of the disclosure, can be prepared using an antibody having one or more of the V_{H}/V_{L} sequences of the antibody of the present disclosure, as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Particularly, conservative modifications (as known in the art) are introduced. The mutations can be amino acid substitutions, additions or deletions, particularly are substitutions. Moreover, typically no more than one, two, three, four or five residues within a CDR region are altered.

Engineered antibodies of the disclosure include those in which modifications have been made to framework residues within V_{H} and/or V_{L}, e.g. to improve the properties of the antibody. Typically, such framework modifications are made to decrease the immunogenicity of the antibody. For example, one approach is to “back-mutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043.

In addition, or as an alternative to modifications made within the framework or CDR regions, the bi-/multi-specific recombinant protein of the disclosure can be engineered to include modifications within the Fe region, typically to alter one or more functional properties of the antibody, such as serum half-life, complement fixation, Fc receptor binding, and/or antigen-dependent cellular cytotoxicity. Furthermore, the bi-/multi-specific recombinant protein of the disclosure can be chemically modified (e.g., one or more chemical moieties can be attached to the antibody) or be modified to alter its glycosylation, again to alter one or more functional properties of the bi-/multi-specific recombinant protein.

In one embodiment, the hinge region of C_{H1} is modified in such that the number of cysteine residues in the hinge region is altered, e.g., increased or decreased. This approach is described further in U.S. Pat. No. 5,677,425. The number of cysteine residues in the hinge region of C_{H1} is altered to, for example, facilitate assembly of the light and heavy chains or to increase or decrease the stability of the antibody.

In another embodiment, the Fc hinge region in the bi-/multi-specific recombinant protein is mutated to decrease the biological half-life of the bi-/multi-specific recombinant protein. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of the bi-/multi-specific recombinant protein is modified. For example, a de-glycosylated bi-/multi-specific recombinant protein can be made (i.e., the bi-/multi-specific recombinant protein lacks glycosylation). Glycosylation can be altered to, for example, increase the affinity of the bi-/multi-specific recombinant protein for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such de-glycosylation may increase the affinity of the antibody for antigen. See, e.g., U.S. Pat. Nos. 5,714,350 and 6,350,861.

Another modification of the bi-/multi-specific recombinant protein herein that is contemplated by this disclosure is pegylation. A bi-/multi-specific recombinant protein can be pegylated to, for example, increase the biological (e.g., serum) half-life of the bi-/multi-specific recombinant protein. To pegylate a bi-/multi-specific recombinant protein, the bi-/multi-specific recombinant protein typically is reacted with polyethylene glycol (PEG), such as a reactive ester or aldehyde derivative of PEG, under conditions in which one or more PEG groups become attached to the bi-/multi-specific recombinant protein. Particularly, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. Methods for pegylating proteins are known in the art and can be applied to the antibodies of the disclosure. See, e.g., EP 0 154 316 and EP 0 401 384.

### Nucleic Acid Molecules

In another aspect, the disclosure provides a nucleic acid molecule that may encode the IL-2 variant or the recombinant protein of the disclosure.

The nucleic acid molecule can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the disclosure can be, e.g., DNA or RNA and may or may not contain intronic sequences.

The nucleic acid molecule of the disclosure can be obtained using standard molecular biology techniques. For example, DNA fragments encoding the CDRs may be operatively linked to those encoding the frameworks. A V_{L}- or V_{H}-encoding DNA fragment is operatively linked to another DNA fragment encoding the constant region. The term “operatively linked”, as used in this context, is intended to mean that the two DNA fragments are joined such that the amino acid sequences encoded by the two DNA fragments remain in-frame.

The isolated DNA encoding the V region can be converted to a full-length heavy chain gene by operatively linking the V_{H}-encoding DNA to another DNA molecule encoding heavy chain constant regions (C_{H1}, C_{H2} and C_{H3}). The sequences of human heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art and DNA fragments encompassing these regions can be obtained by standard PCR amplification.

To create a scFv gene, the V_{H}- and V_{L}-encoding DNA fragments are operatively linked to another fragment encoding a flexible linker, e.g., a GS linker, such that the V_{H} and V_{L} sequences can be expressed as a contiguous single-chain protein, with the VL and VH regions joined by the flexible linker.

DNA fragments encoding the polypeptides of the disclosure may be inserted into one or more expression vectors such that these DNA fragments are operatively linked to transcriptional and translational regulatory sequences. The expression vectors may be transfected or transformed into host cells, to express the polypeptides of the disclosure.

The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The expression vectors may encode a signal peptide that facilitates secretion of the IL-2 variant or the polypeptides of the recombinant protein from a host cell. The DNA fragments encoding the IL-2 variant or the polypeptides may be cloned into the vectors such that the signal peptide is linked in-frame to the amino terminus of said DNA fragments. The signal peptide can be an immunoglobulin signal peptide or a heterologous signal peptide (i.e., a signal peptide from a non-immunoglobulin protein).

The expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Particularly, the selectable marker genes include the dihydrofolate reductase (DHFR) gene (for use in dhfr-host cells with methotrexate selection/amplification) and the neo gene (for G418 selection).

The expression vector(s) may be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the IL-2 variant or the polypeptides of the disclosure in either prokaryotic or eukaryotic host cells, expression of the IL-2 variant or the polypeptides in eukaryotic cells, and particularly mammalian host cells, is the most preferred because such eukaryotic cells, and in particular mammalian cells, are more likely than prokaryotic cells to assemble and secrete a properly folded IL-2 variant or polypeptides of the disclosure.

Examples of expression vectors include but are not limited to plasmids, viral vectors, yeast artificial chromosomes (YACs), bacterial artificial chromosomes (BACs), transformation-competent artificial chromosomes (TACs), mammalian artificial chromosomes (MACs) and human artificial episomal chromosomes (HAECs).

The mammalian host cells for expressing the IL-2 variant or the polypeptides of the disclosure include Chinese Hamster Ovary (CHO cells) (including dhfr-CHO cells, described in Urlaub and Chasm, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841.

### Pharmaceutical Compositions

In another aspect, the disclosure provides a pharmaceutical composition, which may comprise the IL-2 variant, the recombinant protein, the nucleic acid molecule, the expression vector, or the host cell, of the disclosure, formulated together with a pharmaceutically acceptable carrier. The pharmaceutical composition may optionally contain one or more additional pharmaceutically active ingredients, such as an anti-tumor antibody.

The pharmaceutical composition may comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

The pharmaceutical composition may be suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Alternatively, the IL-2 variant or the recombinant protein of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a micro-emulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.010% to about 99% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, the protein of the disclosure can be administered as a sustained release formulation, in which case less frequent administration is required.

The dose of the IL-2 variant or the recombinant protein of the disclosure may be determined by physicians depending on a subject's e.g., sex, age, medical history and etc.

A “therapeutically effective dosage” of the IL-2 variant or the recombinant protein of the disclosure, may result in a decrease in severity of disease symptoms, or an increase in frequency and duration of disease symptom-free periods. For example, for the treatment of tumor-bearing subjects, a “therapeutically effective dosage” may reduce tumor size by at least about 20%, at least about 40%, at least about 60%, and at least about 80%, or even eliminate tumors, relative to untreated subjects.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Pharmaceutical compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the IL-2 variant or the recombinant protein of the disclosure can be formulated to ensure proper distribution in vivo. For example, to ensure that the IL-2 variant or the recombinant protein of the disclosure cross the blood-brain barrier, they can be formulated in liposomes, which may additionally comprise targeting moieties to enhance selective transport to specific cells or organs.

### Uses and Methods

The IL-2 variant or the recombinant protein of the disclosure may be used for in vitro activation of immune cells, particularly exhausted immune cells. The method may comprise contacting an immune cell with the IL-2 variant, the recombinant protein, or the nucleic acid molecule, expression vector or host cell expressing the same, of the disclosure. The immune cell may be a naïve immune cell, or an exhausted immune cell, such as an effector T cells.

The recombinant protein, especially the bi-/tri-specific recombinant protein, of the disclosure may be used to treating cancers, or activating effector immune cells in the tumor environment. Particularly, the application provides a method for treating or alleviating a tumor, or activating an effector immune cell in a tumor microenvironment in a subject in need thereof, comprising administering to the subject the pharmaceutical composition of the disclosure. The pharmaceutical composition may comprise the IL-2 variant, the recombinant protein, or the nucleic acid molecule, the expression vector or the host cell for preparing the same, of the disclosure. The recombinant protein may comprise an IL-2 variant and an antigen binding domain targeting a specified antigen in the tumor lesion site. The specified antigen in the tumor lesion site may be an antigen expressed by a certain cell such as a tumor cell or an immune cell, e.g., carcinoembryonic antigen (CEA), fibroblast activation protein (FAP), PD-1 and the like. Alternatively, the recombinant protein may comprise an IL-2 variant, an antigen binding domain targeting a specified antigen in the tumor lesion site, and an antigen binding domain targeting an antigen expressed by an exhausted immune cell. The antigen expressed by an exhausted immune cell may be e.g., LAG-3 or TIM-3.

The recombinant protein, especially the bi-/tri-specific recombinant protein, of the disclosure may be used to treat or alleviate an autoimmune disease. Particularly, the application provides a method for treating or alleviating an autoimmune disease in a subject in need thereof, comprising administering to the subject the pharmaceutical composition of the disclosure. The pharmaceutical composition may comprise a recombinant protein, or the nucleic acid, expression vector or the host for preparing the same. The recombinant protein may comprise an IL-2 variant and an antigen binding domain targeting a Treg in the autoimmune disease lesion site. The antigen associated with a Treg may be e.g., CD4 or FOXP3.

In certain embodiments, the subject may be a mammal, especially human.

The disclosure provides the use of the IL-2 variant, the recombinant protein, the nucleic acid molecule, the expression vector, the host cell or the pharmaceutical composition of the disclosure in preparation of a medicament for treating or alleviating a tumor or an autoimmune disease, or activating an immune cell in a tumor lesion site.

The IL-2 variant or the recombinant protein of the disclosure may be administered with an additional agent, such as an anti-tumor agent, or an agent against and autoimmune disease.

The combination of therapeutic agents discussed herein can be administered concurrently as a single composition in a pharmaceutically acceptable carrier, or concurrently as separate compositions with each agent in a pharmaceutically acceptable carrier. In another embodiment, the combination of therapeutic agents can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

Although the present application and its advantages have been described in detail, it should be understood that various changes, substitutions and alterations can be made herein without departing from the spirit and scope of the invention as defined in the appended claims.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1. Construction of Cell Lines Stably Expressing PD-1

The cDNA sequence encoding human PD-1 (amino acid sequences set forth in SEQ ID NO: 47) was synthesized, and then sub-cloned into the pLV-EGFP(2A)-Puro vector (Beijing Inovogen, CN). Lentiviruses were generated in HEK293T cells (Cobioer, NJ, CN) by co-transfection of the resultant pLV-EGFP(2A)-Puro-PD-1, psPAX and pMD2.G plasmids, according to the instruction in Lipofectamine 3000 kit (Thermo Fisher Scientific, USA). Three days post co-transfection, the lentiviruses were harvested from the HEK293T cell culture supernatants (the culture medium comprising DMEM (Cat #:SH30022.01, Gibco) with 10% FBS (Cat #:FND500, Excell)), and then used to infect HEK-Blue/IL-2 cells (Invivogen, FR) or HEK293A cells, to generate HEK-Blue/IL-2/PD-1 cells and HEK293A/PD-1 cells. These HEK293A cells were cultured in DMEM (Cat #:SH30022.01, Gibco, USA) comprising 10% FBS (Cat #:FND500, Excell, CN) and 0.2 μg/ml puromycin (Cat #:A11138-03, Gibco) for 7 days. The expressions of the PD-1 molecules were confirmed by FACS using commercially available human anti-PD-1 antibody (PE anti-human PD-1 antibody, Cat #:ab233178, Abeam, USA).

### Example 2. Design of IL-2 Variants

To attenuate IL-2's binding capability to the IL2R a subunit (IL2Rα, also known as CD25), the crystal configuration of the IL-2-CD25 complex (PDB: IZ92) was analyzed.

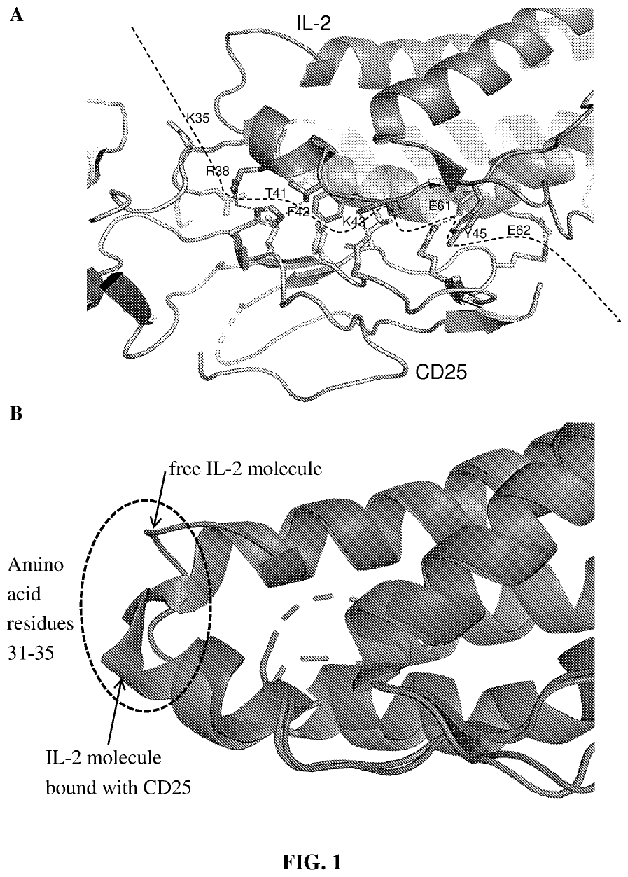

As shown in FIG. 1 (A), the interface between IL-2 and CD25 was divided into the center and the surrounding part. At the center of the interface, the F42 and Y45 residues of IL-2 interacted with the hydrophobic side chain of CD25 via hydrophobic bonding. At the surrounding part of the interface, the K35, R38, T41, K43, E61 and E62 residues in IL-2 formed multiple ionic bonds and hydrogen bonds with CD25. The configuration of a free IL-2 molecule (PDB: 1M4C) was further analyzed in comparison with that of a CD25 bound IL-2 molecule. It was found that during the CD25 binding process, the 31^{st} to 35^{th} amino acid residues of IL-2, i.e., YKNPK, changed a lot in conformation (FIG. 1 (B)), probably because this region was flexible. The conformational change may play a key role in IL-2-CD25 interaction. Therefore, amino acid modifications, including deletions and substitutions, were introduced into this region, to reduce the interaction between the IL-2 molecule and the CD25 molecule.

Table 1 shows the core mutations made to the IL-2 molecule, wherein the wildtype IL-2-wt3 was designed and prepared according to UNIPROT: P60568. The wildtype IL-2 molecule and the IL-2 variants all contained T3A and C125S mutations, to reduce the occurrence of O-glycosylation and count of disulfide bonds, so as to reduce the formation of aggregates.

TABLE 1
Mutations in IL-2 variants

| Protein ID | Core mutations | Protein sequence |
|---|---|---|
| IL2-wt3 | | SEQ ID NO: 3 |
| IL2-G12 | delP34-K35 | SEQ ID NO: 4 |
| IL2-G13 | delP34-K35, F42A | SEQ ID NO: 5 |
| IL2-D22 | delK35, Y45R | SEQ ID NO: 6 |
| IL2-D37 | delK35, F42A, Y45R | SEQ ID NO: 7 |

### Example 3. Construction, Expression and Purification of Fc-IL-2 Recombinant Proteins

Based on the IL-2 design in Example 2, recombinant proteins each containing an immunoglobulin Fe region and an IL-2 peptide were constructed and tested for binding capability. Specifically, a Fc region (SEQ ID NO: 8) was linked via a linker (SEQ ID NO: 9) to the IL-2, as shown in FIG. 2 (A). A positive control (PC) protein was prepared with the same Fc-linker-IL-2 structure using the amino acid sequence of SEQ ID NO: 15, wherein the IL-2 part was from RG6279 (eciskafusp alfa) and published in CN103492411 B as SEQ ID NO: 19.

Table 2 shows the information of the obtained recombinant proteins.

DNA fragments encoding these recombinant proteins were synthesized and cloned into the GS vectors between the ClaI and XhoI restriction sites. The GS vectors were transformed into cells, and clones were picked for sequencing, to obtain expression vectors with correct sequences.

TABLE 2
Recombinant Fc-IL-2 furoin proteins

| Protein ID | IL-2 as used | Protein sequence |
|---|---|---|
| WT | IL2-wt3 | SEQ ID NO: 10 |
| G12 | IL2-G12 | SEQ ID NO: 11 |
| G13 | IL2-G13 | SEQ ID NO: 12 |
| D22 | IL2-D22 | SEQ ID NO: 13 |
| D37 | IL2-D37 | SEQ ID NO: 14 |
| PC | | SEQ ID NO: 15 |

HEK-293F cells (Cobioer, CN) were transfected with the expression vectors obtained above using PEI. Briefly, the HEK-293F cells were transfected with the expression vectors using polyethyleneinimine (PEI) at a DNA:PEI ratio of 1:3, 1.5 μg of DNAs per millimeter. The transfected HEK-293F cells were cultured in an incubator at 37° C. under 5% CO₂ with shaking at 120 RPM. After 10-12 days, the cell culture supernatants were harvested, centrifuged at 3500 rpm for 5 min, and flowed through a 0.22 m film filter to remove the cell debris. The recombinant proteins as expressed were purified using pre-equilibrated Protein-A affinity columns (Cat #:17040501, GE, USA) and eluted with the elution buffer (20 mM citric acid, pH 3.0-3.5). The buffer was replaced in an ultrafilter, and the obtained fusion proteins were kept in PBS (pH 7.4) and measured for the concentrations using a NanoDrop analyzer.

### Example 4. Binding Activity of Recombinant Fc-IL-2 Proteins to CD25

The purified recombinant Fc-IL-2 proteins were tested for their binding capability to the recombinant human CD25 protein.

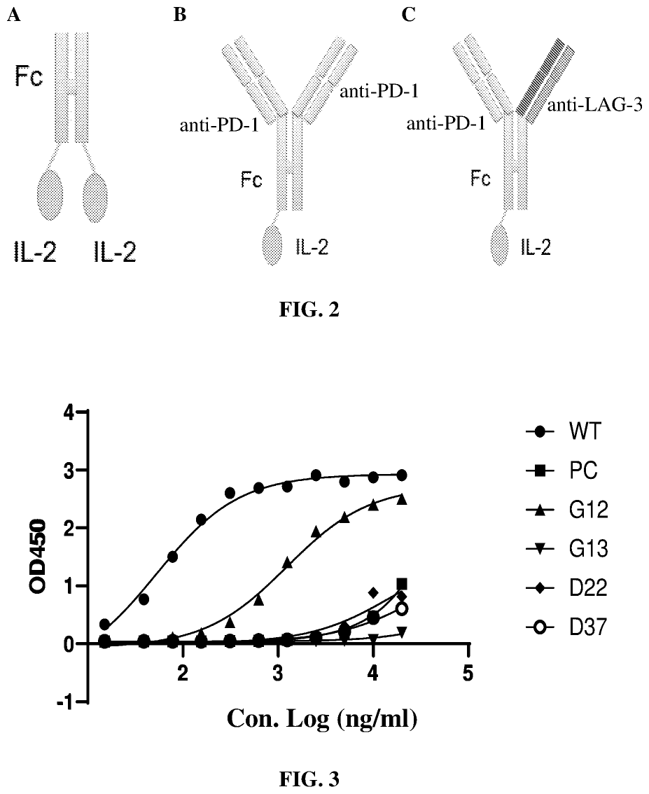

Briefly, 1 μg/mL recombinant CD25 proteins (Cat #: ILA-H52H9, ACRO, CN) diluted in PBS, were added to 96-well ELISA plates, 100 μl per well. The ELISA plates were kept still at 4° C. overnight, blocked with 250 μl PBST with 4% skimmed milk powder at room temperature for 1 h, washed by 200 μl PBST for three times, added with 100 μl serially diluted recombinant Fc-IL-2 proteins, 2-fold dilution starting at 20 μg/mL, and incubated at 37° C. for 1 h, wherein PBST was added to the negative control well. The ELISA plates were washed with 200 μl PBST for three times, added with 100 μl HRP-goat-anti-human IgG antibodies (1:7000 diluted, Cat #: 31413, thermo-fisher), and incubated at room temperature for 1 h. Then, the plates were washed with 200 μl PBST for four times, added with 100 μl TMB (Cat #: 555214, BD) followed by 50 μl 10% H₂SO₄, and measured for absorbance at 450 nm. The data were processed and analyzed using GraphPad, and the results were shown in FIG. 3.

According to FIG. 3, the recombinant Fc-IL-2 protein WT containing the wildtype IL-2 showed potent CD25 binding capability, while the recombinant Fc-IL-2 proteins containing the IL-2 variants, including G13, D22 and D37, as well as the positive protein PC, barely bound CD25.

### Example 5. Binding Capability of Recombinant Fc-IL-2 Proteins to Tregs and CD8⁺ Effector T Cells

The Tregs constitutively express IL-2Rαβγ, i.e., the high-affinity IL-2 receptor, at high levels, while the NK cells and the CD8⁺ effector cells mainly express IL-2Rβγ.

The recombinant Fc-IL-2 proteins of the disclosure were tested for their binding ability to primary human Tregs (Allcells, US) and CD8⁺ effector T cells (Allcells, US). Briefly, the Tregs or CD8⁺ effector T cells were centrifuged at 1000 g for 5 min, washed by the washing solution (1×PBS+2% FBS) once, and diluted to cell density of 2×10⁶/ml. The recombinant Fc-IL-2 proteins of the disclosure were diluted, and added to plates in 50 μl/well. The plates were added with 50 μl/well of the cells prepared above, and incubated on ice for 60 min, with the final concentration of the recombinant Fc-IL-2 protein in the plates at 10 μg/ml, 1 μg/ml, 0.1 μg/ml and 0.01 μg/ml, respectively. The plates were added with 200 μl/well of washing solution, centrifuged at 300 g for 5 min, and the supernatants were discarded. The washing-centrifugation procedure was repeated twice. The cells were added with 1:500 diluted PE F(ab′)₂ goat-anti-human IgG(γ) antibodies (Cat #: 398004, BioLegen), 50 μl per well, and incubated on ice for 40 min. The cells were subjected to the flow cytometry, and data were analyzed using GraphPad.

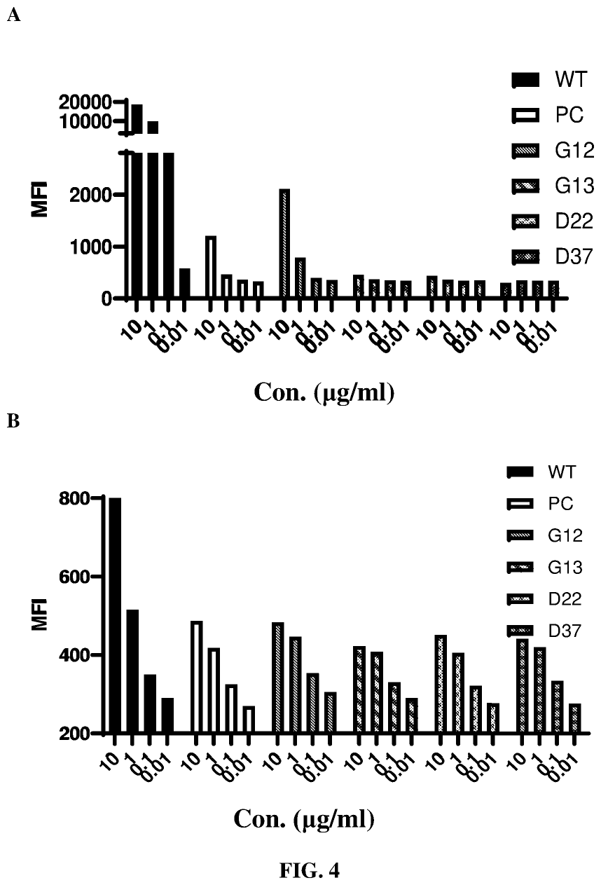

The results were shown in FIG. 4. The recombinant Fc-IL-2 protein WT bound the Tregs with a high activity, while the recombinant Fc-IL-2 proteins containing the IL-2 variants, as well as the positive control protein, showed binding activity reduction to some extent. Particularly, G13, D22 and D37 showed lower Treg binding capability than the positive control (FIG. 4 (A)), suggesting the elimination of these recombinant Fc-IL-2 proteins' biased binding to Tregs. In the meanwhile, the recombinant Fc-IL-2 proteins containing the IL-2 variants showed a bit decreased binding capability to CD8⁺ effector T cells compared to WT but comparable to that of the positive control (FIG. 4 (B)).

The results illustrated that the Fc-IL-2 proteins G13, D22 and D37 maintained their binding capability to effector T cells, with their biased binding capability to Tregs significantly reduced or eliminated.

### Example 6. Activity of Recombinant Fc-IL-2 Proteins to Trigger IL-2 Signaling Pathways

The recombinant Fc-IL-2 proteins were measured for their ability to activate the signaling pathways in HEK-Blue/IL-2 report cells expressing human IL-2Rα, IL-2Rβ, and IL-2Rγ.

Briefly, the HEK-Blue/IL-2 cells (InvivoGen, US) were cultured in DMEM medium (Cat #: SH30243.01, Hyclone, US) containing 10% FBS (Cat #: FND500, Excell, CN), 10 μg/ml puromycin (Cat #: A11138-03, GIBCO, US), 100 μg/ml Normocin™ (Cat #: ant-nr-2, Invivogen, US), and 100 μg/ml bleomycin (Cat #: ant-Zn-5, Invivogen, US). Then, 4×10⁴ HEK-Blue/IL-2 cells in 200 μl cell culture medium were plated onto 96-well plates, and incubated at 37° C. overnight (about 12 h). The cell culture medium was replaced with 200 μl fresh pure DMEM medium without addition of additional components, and the cells were cultured for another 7 h. The DMEM culture medium in each well was replaced with 100 μl HEK Blue detection buffer (Cat #: hb-det3, Invivogen, US) containing the recombinant Fc-IL-2 protein of the disclosure at different concentrations ranging from 100 μg/ml to 0.01 ng/ml. The cells were cultured at 37° C. until the culture medium turned blue. The absorbance at 630 nm was read in a microplate reader (SpectraMaxR i3X, Molecular Devices, US).

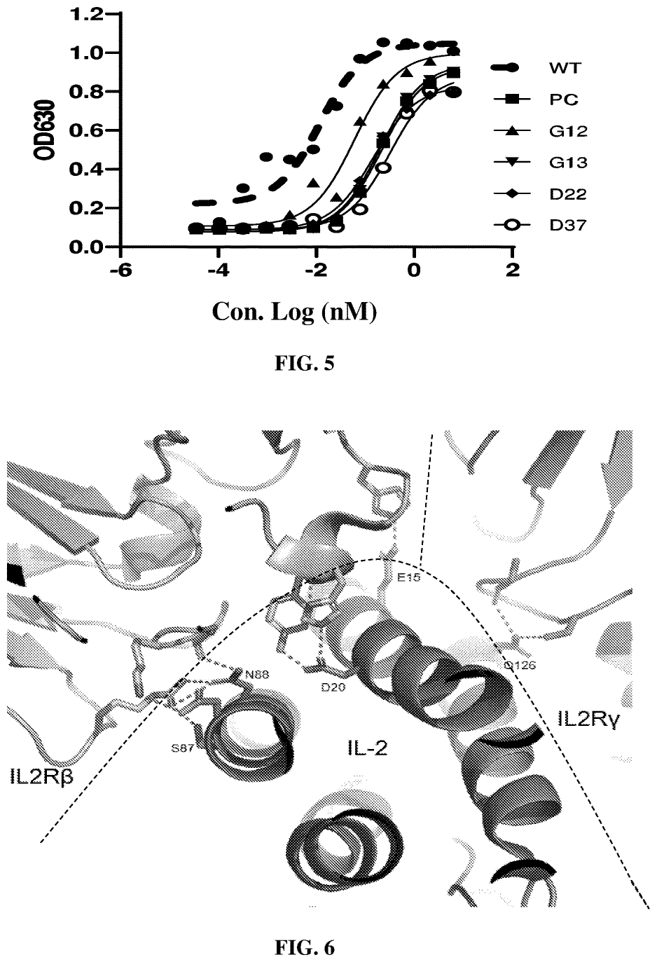

The EC₅₀ values were summarized in Table 3, and the binding curves of the representative recombinant Fc-IL-2 proteins were shown in FIG. 5. It can be seen that the ability of the recombinant Fc-IL-2 proteins of the disclosure to activate the HEK-Blue/IL-2 cells was reduced. Particularly, the EC₅₀ value of D37 was higher than that of the positive control, suggesting that D37's bias toward cells expressing the high-affinity IL-2 receptor was somewhat decreased or eliminated.

TABLE 3
Activity of recombinant Fc-IL-2 proteins
to activate HEK-Blue/IL-2 cells

| | | EC₅₀ ratio of IL-2 variant- |
|---|---|---|
| Protein ID | EC₅₀ (nM) | containing protein to WT |
| WT | 0.012 | 1 |
| G12 | 0.060 | 5 |
| G13 | 0.204 | 17 |
| D22 | 0.145 | 12 |
| D37 | 0.321 | 26.7 |
| PC | 0.221 | 18 |

### Example 7. Mutation Optimization in IL-2 Variants

Based on the crystal structure of the complex formed by the IL-2 with IL2Rβ and IL2Rγ (PDB: 2B51), IL-2 variants with further reduced binding affinity to the IL-2 receptors were designed. According to FIG. 6, the amino acid residues in the IL-2, including E15, D20, D84, S87 and N88, formed salt bridges or hydrogen bonds with the IL2Rβ subunit, and Q126 interacted with the IL2Rγ subunit via a hydrogen bond.

The amino acid residues mentioned above were mutated in D37, to remove the salt bridges or hydrogen bonds and thus further reduce the protein's ability to active the IL-2 signaling pathways. The mutations and created IL-2 variants were set forth in Table 4.

TABLE 4
Mutations in D37

| Protein ID | Core mutations | Protein sequence |
|---|---|---|
| D37-1 | delK35, F42A, Y45R, E15Q | SEQ ID NO: 16 |
| D37-3 | delK35, F42A, Y45R, S87T | SEQ ID NO: 17 |
| D37-4 | delK35, F42A, Y45R, N88D | SEQ ID NO: 18 |
| D37-5 | delK35, F42A, Y45R, Q126T | SEQ ID NO: 19 |
| D37-6 | delK35, F42A, Y45R, D20L | SEQ ID NO: 20 |

### Example 8. Preparation of Bispecific Recombinant Proteins

The IL-2 variants as designed in Example 7 were used for construction of bispecific recombinant proteins each further containing an anti-PD-1 antibody, whose heavy chain and light chain variable region sequences were from Keytruda® and set forth in SEQ ID NOs: 27 and 28, respectively.

The bispecific recombinant proteins were prepared in an asymmetric manner, i.e., each of such proteins contained two PD-1 binding domains and one IL-2 molecule, and referred to as 309 molecules herein. The details of the bispecific recombinant proteins can be found in Table 5 and FIG. 2 (B). The molecule 309-0 contained a half-antibody 309 without any IL-2 molecule, and a half-antibody 309-0 with an IL-2 molecule linked at the C terminus of the heavy chain. Similarly, the bispecific recombinant proteins 309-1, 309-3, 309-4, 309-5 and 309-6 all contained a half-antibody 309, and respectively contained a half-antibody 309-1, 309-3, 309-4, 309-5 and 309-6 each with an IL-2 molecule linked at the C terminus of the heavy chain. The half-antibody 309 contained a hole mutation at the heavy chain constant region, and the other half-antibodies each contained a knob mutation at the heavy chain constant region. All half-antibodies contained the same light chain.

TABLE 5
Bispecific recombinant proteins

| Molecule | IL-2 as | Sequences used in the bispecific recombinant |
|---|---|---|
| ID | used | proteins |
| 309-0 | IL2-D37 | Half-antibody 309 contained a heavy chain of |
| | | SEQ ID NO: 29 and a light chain of SEQ ID |
| | | NO: 30, |
| | | Half-antibody 309-0 contained a long chain of |
| | | SEQ ID NO: 31 and a short chain of SEQ ID |
| | | NO: 30. |
| 309-1 | IL2-D37-1 | Half-antibody 309, see above, |
| | | Half-antibody 309-1 contained a long chain of |
| | | SEQ ID NO: 32 and a short chain of SEQ ID |
| | | NO: 30. |
| 309-3 | IL2-D37-3 | Half-antibody 309, see above, |
| | | Half-antibody 309-3 contained a long chain of |
| | | SEQ ID NO: 33 and a short chain of SEQ ID |
| | | NO: 30. |
| 309-4 | IL2-D37-4 | Half-antibody 309, see above, |
| | | Half-antibody 309-4 contained a long chain of |
| | | SEQ ID NO: 34 and a short chain of SEQ ID |
| | | NO: 30. |
| 309-5 | IL2-D37-5 | Half-antibody 309, see above, |
| | | Half-antibody 309-5 contained a long chain of |
| | | SEQ ID NO: 35 and a short chain of SEQ ID |
| | | NO: 30. |
| 309-6 | IL2-D37-6 | Half-antibody 309, see above, |
| | | Half-antibody 309-6 contained a long chain of |
| | | SEQ ID NO: 36 and a short chain of SEQ ID |
| | | NO: 30. |

The DNA fragments encoding the long (heavy) chains (each containing the variable region and the constant region) and the short (light) chains (each containing the variable region and the constant region) were synthesized. The DNA fragments encoding the short (light) chains were digested with C1aI and HindIII, the DNA fragments encoding the long (heavy) chain were digested with EcoRI and XhoI. The pCMV-plasmids were digested with HindIII and EcoRI to get the promoter fragments, and the GS-vectors were digested using ClaI and XhoI. The DNA fragments were recovered, ligated, transfected into cells, and picked out for sequencing. The expression vectors with correct sequences were obtained, and used to express the bispecific proteins in a single-cell manner.

The HEK-293F cells were transfected with the expression vectors using polyethyleneinimine (PEI) at a DNA:PEI ratio of 1:3, 1.5 jag of DNAs per millimeter. The transfected HEK-293F cells were cultured in an incubator at 37° C. under 50% CO₂ with shaking at 120 RPM. After 10-12 days, the cell culture supernatants were harvested, centrifuged at 3500 rpm for 5 min, and flowed through a 0.22 jam film filter to remove the cell debris. The bispecific recombinant proteins as expressed were purified using pre-equilibrated Protein-A affinity columns (Cat #:17040501, GE, USA) and eluted with the elution buffer (20 mM citric acid, pH 3.0-3.5).

The bispecific recombinant proteins were purified using the anion exchange chromatography. The bispecific recombinant proteins collected from the Protein-A affinity columns were concentrated with a 30 kDa ultrafilter, exchanged for a low-salt buffer (pH 5.5) with a volume of 10 mL, and flowed through a 0.2 m film filter. The anion exchange columns were balanced with low-salt acetate buffer (pH5.5), and loaded with the bispecific recombinant protein samples. The anion exchange columns were balanced with low-salt acetate buffer (pH5.5), and elution was done using 0-100% high-salt acetate buffer (pH5.5). The eluates were collected, exchanged for the buffer, kept in PBS (pH7.4), and measured for the molecule concentrations in a NanoDrop analyzer. The bispecific recombinant proteins were subjected to the size exclusion chromatography (SEC) and MS analysis, and confirmed to have protein purity above 90%.

### Example 9. Binding Capability of Bispecific Recombinant Proteins to Human IL-2Rβγ

The bispecific recombinant proteins were measured for their binding affinity to human IL-2Rβγ by BIAcore™ 8K (GE Life Sciences, USA) using the Wizard mode. Briefly, mouse-anti-human Fe antibodies (Cat #: BR100839, cytiva) were coupled to CM5 biosensor chips, and 1 μg/mL bispecific recombinant proteins of the disclosure in HBS-EP buffer (Cat #: BR-1006-69, GE Life Sciences) were flowed through the chip to allow 100 RU (response unit) of the bispecific recombinant proteins being captured by the mouse-anti-human Fe antibodies. The recombinant IL-2Rβγ proteins (Cat #: ILG-H5283, ACRO) at the concentration of 1, 0.5, 0.25, 0.125, 0.0625, 0.03125, 0.015625, 0.0078125, or 0.003900625 g/ml in HBS-EP buffer, were respectively flowed through the chip. RG6279, the bispecific recombinant protein containing an anti-PD-1 antibody and an IL-2 molecule, was prepared using the sequences disclosed in WO2018184964A1 as SEQ ID NOs: 22, 23 and 25 with the structure in FIG. 2 (B), and used as the positive control.

The data were processed in the BIA evaluation software, and K_{D} values were determined and summarized in Table 6. It can be seen that the bispecific recombinant proteins of the disclosure all bound IL-2Rβγ but with different binding affinity, wherein the binding affinity of 309-4, 309-5 and 309-6 was significantly decreased and evidently lower than that of the RG6279 analogue.

TABLE 6
Binding affinity of bispecific recombinant proteins to IL-2Rβγ

| Protein ID | ka (1/Ms) | kd (1/s) | K_{D} (M) |
|---|---|---|---|
| 309-0 | 1.92E+05 | 9.16E−04 | 4.77E−10 |
| 309-1 | 1.45E+05 | 1.25E−04 | 8.60E−10 |
| 309-3 | 1.52E+05 | 9.42E−05 | 6.20E−10 |
| 309-4 | 1.38E+05 | 3.31E−04 | 2.40E−09 |
| 309-5 | 1.16E+05 | 4.28E−04 | 3.70E−09 |
| 309-6 | 1.35E+05 | 7.63E−03 | 5.66E−08 |
| RG6279 | 2.10E+05 | 6.61E−05 | 3.15E−10 |
| analogue | | | |

### Example 10. Ability of Bispecific Recombinant Proteins to Activate IL-2 Signaling Pathways

The bispecific recombinant proteins of the disclosure were tested for their capability to induce the IL-2 signaling, using the PD-1⁻ HEK-Blue/IL-2 cells, according to the protocol of Example 5.

The results were shown in FIG. 7 (A) and Table 7. The capability of bispecific proteins 309-1 and 309-3 to trigger IL-2 signaling was similar to that of 309-0 and the RG6279 analogue, while 309-4, 309-5 and 309-6's activity on IL-2 signaling activation was significantly reduced, almost 1000-fold lower than that of 309-0 and the RG6279 analogue. Particularly, no IL-2 signaling was observed on the cells treated with the molecule 309-6, whose activity to activate IL-2 signaling was similar to that of the Keytruda® analogue, the anti-PD-1 antibody used the negative control.

The bispecific proteins 309-4, 309-5 and 309-6 were further tested using the PD-11⁺ HEK Blue/IL-2 report cells prepared in Example 1, to see whether more bispecific recombinant proteins of the disclosure would be recruited to the PD-1⁺ HEK Blue/IL-2 cells via the interaction between the PD-1 binding domains in the bispecific recombinant proteins and the PD-1 molecules on PD-1V HEK Blue/IL-2 cells, so as to promote the IL-2 signaling activation in a cis-activation manner.

The results were shown in FIG. 7 (B) and Table 7. The three bispecific recombinant proteins of the disclosure showed capability to induce IL-2 signaling in PD-1⁺ HEK Blue/IL-2 cells. The difference between these bispecific recombinant proteins' reaction to PD-1⁻ HEK-Blue/IL-2 cells and to PD-1⁺ HEK-Blue/IL-2 cells, as measured by the ratio of respective EC₅₀ values, suggested that these bispecific recombinant proteins were likely to be safer than the RG6279 analogue as a medicament, providing a wider therapeutic window.

TABLE 7
Capability of bispecific recombinant proteins to induce IL-2 signaling
in PD-1⁻ and PD-1⁺ HEK Blue/IL-2 cells

| | | EC₅₀ (ng/mL)- | |
|---|---|---|---|
| Molecule | EC₅₀ (ng/mL)- | PD1⁺ HEK | PD1⁻ EC₅₀/PD1⁺ |
| ID | PD1⁻ HEK-Blue/IL-2 | Blue/IL-2 | EC₅₀ |
| 309-0 | 5.343 | 0.06061 | 89 |
| 309-1 | 11.55 | — | |
| 309-3 | 6.561 | — | |
| 309-4 | 11572 | 0.1705 | 67870 |
| 309-5 | 9677 | 6.373 | 1518 |
| 309-6 | N/A | 166.1 | >>1000 |
| RG6279 | 7.166 | 0.007818 | 916 |
| analogue | | | |
| Keytruda ® | N/A | N/A | |
| analogue | | | |

### Example 11. Binding Capability of Bispecific Recombinant Proteins to PD-1

The bispecific recombinant proteins of the disclosure were measured for their binding capability to the PD-1-expressing HEK293A cells prepared in Example 1 by FACS.

Briefly, 96-well plates were plated with 10⁵ HEK293/PD-1 cells in 50 μl PBS, and added with 100 μl serially diluted bispecific recombinant proteins of the disclosure or the Keytruda® analogue, the highest concentration being 40 μg/ml. After 1-h incubation at 4° C., the plates were washed by PBST for three times, added with 1:500 diluted APC-goat-anti-mouse IgG antibodies (Cat #: 405308, BioLegen, US). After 1-h incubation at 4° C., the plates were washed by PBST for three times, and subjected to FACS in a flow cytometer (BD).

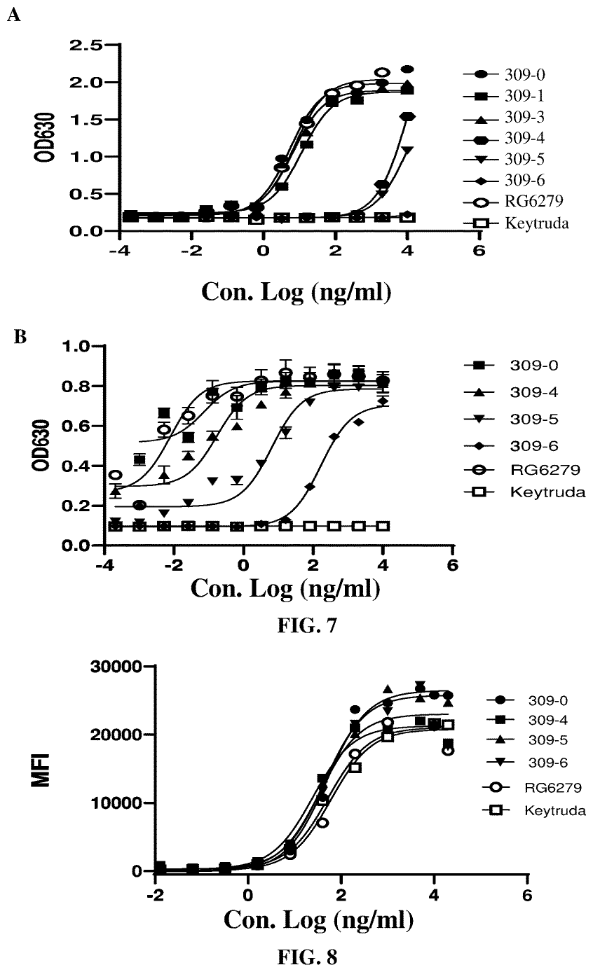

The results were shown in FIG. 8. All the bispecific recombinant proteins of the disclosure bound to the human PD-1 protein, with their binding ability comparable to that of the Keytruda® analogue, suggesting that the PD-1 binding domain functioned properly in the bispecific recombinant proteins.

### Example 12. Ability of Bispecific Recombinant Proteins to Induce CD3⁺ T Cell Activation

The bispecific recombinant proteins 309-4, 309-5 and 309-6 were tested for their effect on CD3⁺ T cell activation and proliferation.

Briefly, microplates were coated with 100 μl 5 μg/ml F(ab′)2-goat-anti-human IgG Fe gamma (Cat #: 31163, invitrogen) overnight at 4° C. The plates were washed with PBS twice, added with 100 μl 0.25 μg/ml anti-CD3 antibodies (OKT3, Cat #: ab86883, Abeam), and incubated for 2 h at 37° C. PBMCs from a healthy human donor' blood sample were collected by density gradient centrifugation, and CD3+ T cells were isolated from the PBMCs using Invitrogen Dynabeads Untouched Human CD3⁺ T Cells Kit (Cat #: 11365D, Thermal Fisher Scientific, US), following the kit's manual. The isolated CD3⁺ T cells were labeled with carboxyfluorescein diacetate succinimidyl ester (CFSE) using the CellTrace™ CFSE Cell Proliferation Kit (Cat #: C345541, Invitrogen), following the kit's manual with a bit modification, i.e., the cells were incubated with 2.5 μM CFSE for 10 min at 37° C. The CFSE labeled cells were re-suspended in RIPM1640 medium with 10% FBS at the cell density of 6×10⁵/ml. The plates coated with the anti-CD3 antibodies were added with 100 μl CD3⁺ T cells, and 100 μl serially diluted recombinant proteins of the disclosure, 3-fold dilution starting at 3 μg/ml. The cells were cultured in a CO₂ incubator for 96 h, harvested, washed by PBS for three times, added with 2 μl PE-mouse-anti-human CD69 antibodies (Cat #: 555531, BD, US) and 2 μl BV605 mouse-anti-human CD25 antibodies (Cat #: 562660, BD, US), and incubated at room temperature for 30 min. The cells were centrifuged, washed with PBS for three times, and subjected to FCAS. The CFSE fluorescence intensity was used to assess the cell proliferation rate, and the ratio of CD69+CD25⁺ T cells to the CFSE positive T cells was used to determine the percent of activated T cells.

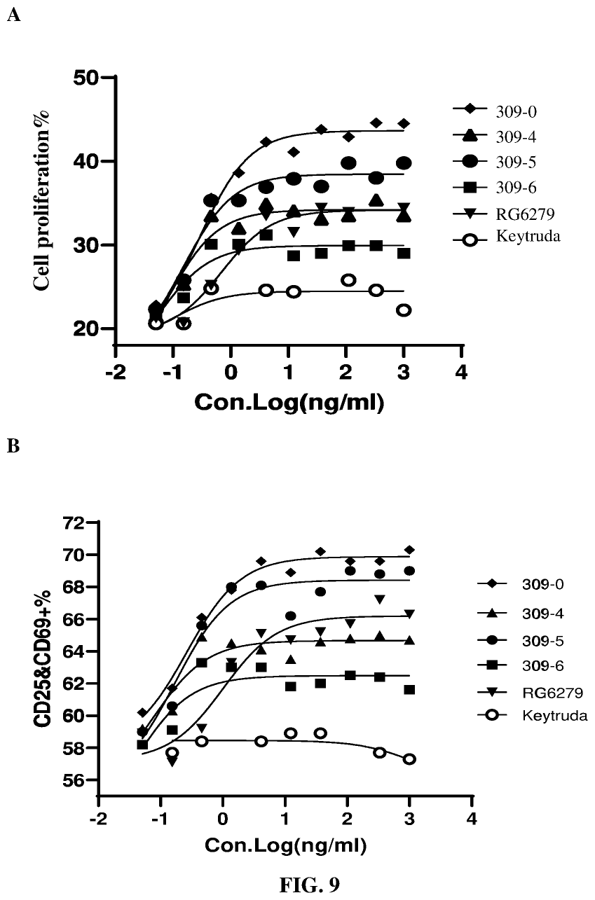

The results were shown in FIG. 9 (A-B). It can be seen that the bispecific recombinant proteins of the disclosure promoted CD3⁺ T cell proliferation and activation as compared to the Keytruda® analogue, suggesting they were able to induce the downstream IL-2 signaling pathways.

### Example 13. In Vivo Anti-Tumor Efficacy of Bispecific Recombinant Proteins in Colon Cancer Xenograft Model

The bispecific recombinant proteins of the disclosure were tested for their in vivo anti-tumor efficacy using the C57BL/6J-mice (also called B6-hPD1 mice, Strain NO. T003095, GemPharmatech, Nanjing, China), a transgenic mice expressing human PD-1 molecules, implanted with MC38-hPD-L1 cells (murine colon carcinoma cells).

Briefly, MC38-hPD-L1 cells, which were genetically engineered to over-express human PD-L1 molecules and have the mouse PD-L1 gene knocked out, were subcutaneously injected into each female C57BL/6J mouse at the right flank on Day 0. These mice were randomly allocated into 6 groups, 6 mice per group, when the average tumor size reached 109 mm³ on Day 6. On Day 6, 13, 16, 20, 23 and 27, the mice from the 6 groups were intraperitoneally administered with PBS, Keytruda® analogue+IL-2 (IL-2wt1, SEQ ID NO: 1) (1.0 mg/kg+0.1 mg/kg), RG6279 analogue (1.0 mg/kg), 309-4 (1.0 mg/kg), 309-5 (1.0 mg/kg) and 309-6 (1.0 mg/kg), respectively. Tumor sizes and mouse body weights were measured twice a week. At the end of the test, mice were sacrificed, and the tumors were collected and photographed. The tumor growth inhibition rate TGI_{TV} (%) ((1−T/C)×100%, T/C=average RTV in administration group/average RTV in control group, RTV refers to the ratio of the tumor volume at a certain day to the tumor volume at Day 6) was calculated and analyzed statistically.

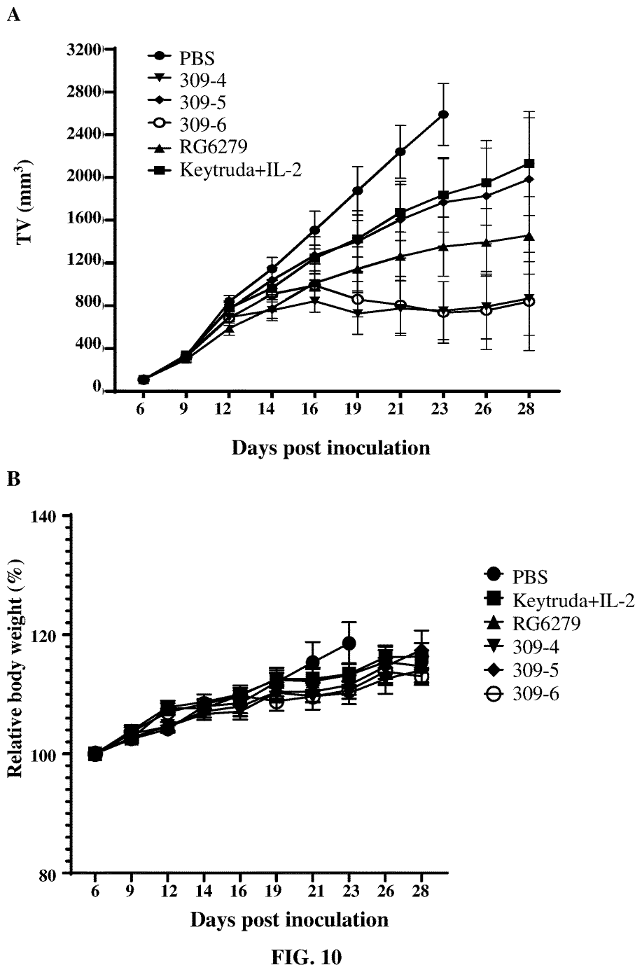

As shown in FIG. 10 (A), when compared to the vehicle control group, the TGIs in the Keytruda® analogue+IL-2 group, the RG6279 analogue group, the 309-4 group, the 309-5 group and the 309-6 group were 28%, 51%, 73%, 31% and 71%, respectively, on Day 23. That is, the tumor growth inhibition effect was significantly better in the administration groups than the vehicle group (p<0.05). Particularly, 309-4 and 309-6 produced the best anti-tumor effect, much better than the Keytruda® analogue+IL-2 combination and also the RG6279 analogue.

The mouse body weight changes during the test were shown in FIG. 10 (B). The mice in the 309-4 group and the 309-6 group had their body weight increased gradually, which was not significantly different from other groups, meaning these two bispecific recombinant proteins were well tolerated and highly safe.

### Example 14. In Vivo Anti-Tumor Efficacy of Bispecific Recombinant Proteins in Pan02 Xenograft Model

The bispecific recombinant proteins of the disclosure were further tested for their in vivo anti-tumor efficacy in the Pan02 xenograft model.

Pan02-hPD-L1 cells, in house prepared by introducing vectors over-expressing human PD-L1 molecules into Pan02 cells (pancreatic cancer cell line), were subcutaneously injected into female C57BL/6J mice engineered to express human PD-1 molecules (Cat #:131083, BIOCYTOGEN) at both the left and right flanks. These mice were randomly allocated into 3 groups, 5 mice per group, when the average tumor size reached 100 mm³, and this day was designated as Day 0. On Day 0, 4, 7, 11, 14, the mice from the 3 groups were intraperitoneally administered with PBS, RG6279 analogue (5.0 mg/kg) and 309-6 (5.0 mg/kg), respectively. Tumor sizes and mouse body weights were measured every two days. Mice were sacrificed when tumor sizes reached 4000 cm³ or at the end of the test, and the tumors were collected and photographed. For each mouse, the average size of the two tumors at the left and right sides was used for data analysis.

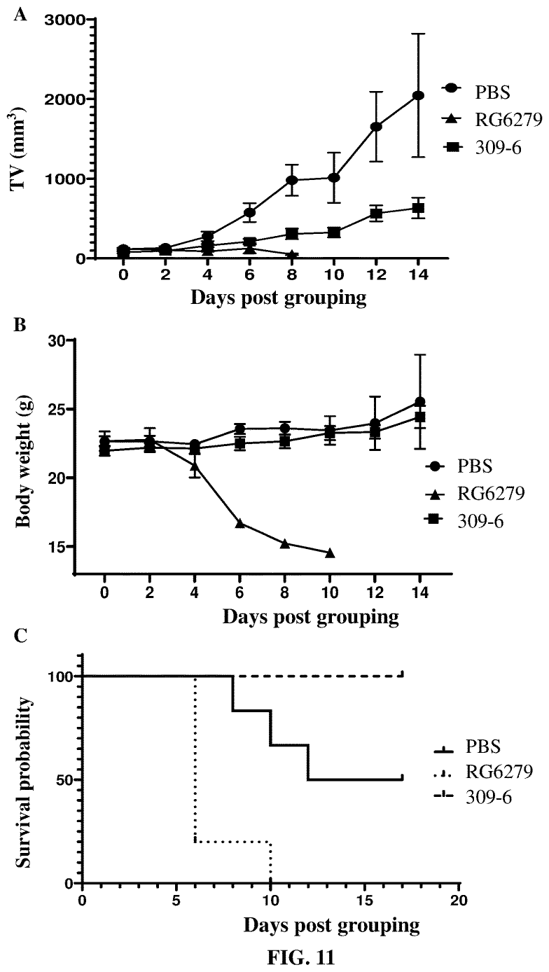

The results were shown in FIG. 11 (A-C). When compared to the vehicle control group, 309-6 exhibited strong antitumor activity, with no mouse weight loss or death observed at the current dose. Although the RG6279 analogue showed better antitumor activity than 309-6, drastic weight loss and/or death was caused by the RG6279 analogue at the specified dose. In conclusion, 309-6 showed a favorable safety profile and high anti-tumor activity in vivo.

### Example 15. Pharmacokinetic of Bispecific Recombinant Proteins

To define major pharmacokinetics (PK) parameters, the plasma concentration of the bispecific recombinant proteins of the disclosure was measured in mice.

Briefly, 9 hPD-1 transgenic C57BL/6J mice (Strain NO. T003095, GemPharmatech, Nanjing, China) were divided into 3 groups and intravenously injected with 1.0 mg/kg Keytruda analogue, 1.0 mg/kg RG6279 analogue, and 1.0 mg/kg 309-6, respectively. About 100 μl of blood was collected from the tail tip of each mouse at 2 h, 24 h, 48 h, 72 h, 96 h, 120 h and 144 h post protein injection, and serum was prepared from each blood sample via centrifugation. The serum was 1:50 diluted with PBS and the protein concentration was measured by ELISA using the human PD1 protein (Cat #:10377-H08H, SINO BIO) to capture the recombinant protein.

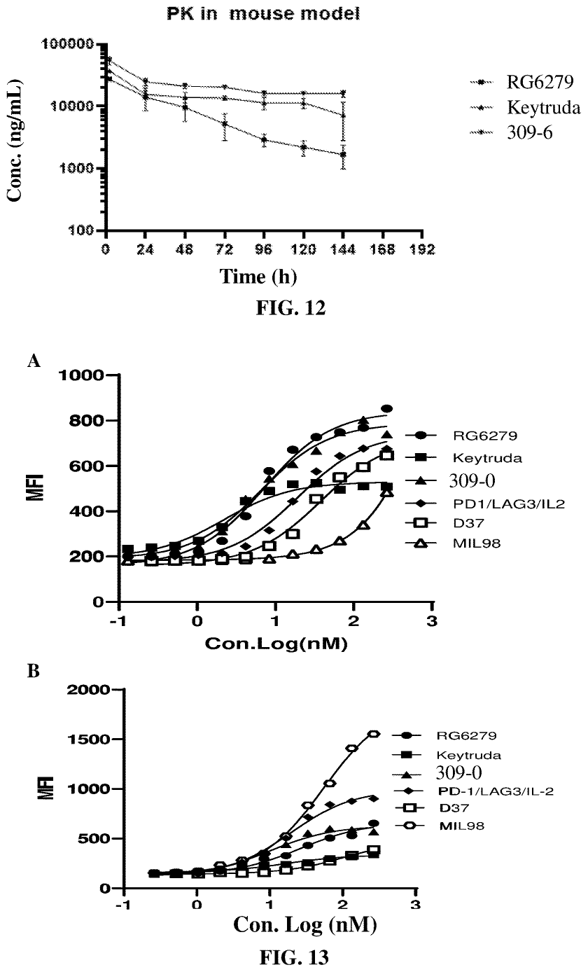

The results were shown in FIG. 12. The bispecific recombinant protein 309-6 exhibited a similar serum profile to the Keytruda analogue. Obviously, 309-6 had a longer serum half-life in mice than the RG6279 analogue.

### Example 16. Construction and Characterization of Tri-Specific Recombinant Proteins

A tri-specific recombinant protein containing IL-2-D37, an anti-PD-1 antibody and an LAG-3 antibody was constructed and characterized. The structure of this tri-specific recombinant protein was shown in FIG. 2 (C), wherein the anti-PD-1 antibody used the heavy and light chain variable region sequences of Keytruda®, and the anti-LAG3 antibody used the heavy chain and light chain variable sequences of SEQ ID NO: 43 and 44 (MIL98 to Mabworks).

Specifically, in the tri-specific recombinant protein, the half-antibody containing the anti-PD-1 antibody and the IL-2 molecule used the half-antibody 309-0, and the half-antibody containing the anti-LAG3 antibody contained a heavy chain and a light chain of SEQ ID NOs: 45 and 46, respectively. The expression vector construction, molecule expression and purification were performed following the protocol in Example 8, with some modifications. Specifically, the two half-antibodies were prepared using a dual-cell expression system, purified and mixed in a 1:1 molar ratio for the assembly. The mixture was added with basic Tris buffer until the pH value reached 8.0, added with a solution containing Glutathione reduced, and stirred at a low speed overnight at 25° C. The mixture was then added with 2 M acetic acid solution until the pH value reached 5.5, and the glutathione reduced was removed via ultrafiltration. The assembled molecules were purified using anion exchange chromatography and cation exchange chromatography. The anion exchange columns were balanced with low-salt Tris buffer (pH8.0), and loaded with the samples. The components that had passed through the columns were collected, and rinsed by low-salt Tris buffer (pH8.0) until UV280 trended to the baseline. The collected samples were adjusted to pH5.5 using an acetic acid solution, concentrated to 1 ml using a 30 kDa ultrafilter tube, and filtered using 0.2 m membrane. The cation exchange columns were balanced with a low-concentration acetate buffer (pH5.5), and loaded with the samples. The low-concentration acetate buffer (pH5.5) was used to balance the columns, and elution was done using 20 CV acetate solutions (concentration at 0-100%, pH5.5). The MS data of the purified molecules revealed protein purity above 90%.

CD3⁺ T cells were isolated from human PBMCs following the protocol of Example 12, and the binding capability of 309-0, the tri-specific recombinant protein of the disclosure, the Keytruda® analogue, and the anti-LAG-3 antibody MIL98, and D37 to the freshly isolated primary CD3⁺ T cells were measured, following the protocol of Example 5.

In addition, the freshly isolated CD3⁺ T cells were re-suspended in 1640 medium with 10% FBS, Glumax and penicillin/streptomycin, at the cell density of 1×10⁶/ml, added with 20 μl Dynabeads™ Human T-Activator CD3/CD28 for T Cell Expansion and Activation (CAT #: 11132D, Gibco, US), and incubated at a 37° C. CO₂ incubator for 9 days. When the cell density was higher than 3×10⁶/ml, the cells were harvested and cultured in new culture vessels with fresh growth medium at the cell density of 1×10⁶/ml. The cells were harvested, and washed by PBS for three times. Then, 309-0, the tri-specific recombinant protein of the disclosure, the Keytruda® analogue, and the anti-LAG-3 antibody MIL98, and D37's binding capability to such exhausted (or over-activated) CD3⁺ T cells were tested, following the protocol of Example 5.

The results were shown in FIG. 13 (A-B). As the LAG-3 expression levels were low in the freshly isolated naïve CD3⁺ T cells, the tri-specific recombinant protein of the disclosure showed lower binding capability to such cells than 309-0. The LAG-3 levels on the exhausted T cells were significantly increased, and the binding activity of MIL98 and the tri-specific recombinant protein of the disclosure to such T cells was evidently increased. Particularly, the tri-specific recombinant protein of the disclosure showed much higher binding capability than 309-0 to the over-activated T cells. In this respect, the tri-specific recombinant protein of the disclosure might be more potent in re-activating the exhausted T cells than the bi-specific molecule.

The sequences in the present application are summarized in Table 8 below.

TABLE 8
Sequences
Description/
Sequence/SEQ ID NO.

IL2-wt1
APTSSSTKKTQLQLEHLLLDLQMILNGINNYKNPKLTRMLTFKFYMPKKATELKHLQCLEEEL
KPLEEVLNLAQSKNFHLRPRDLISNINVIVLELKGSETTFMCEYADETATIVEFLNRWITFCQ
SIISTLT (SEQ ID NO: 1)
IL2-wt2
APTSSSTKKTQLQLEHLLLDLQMILNGINNYKNPKLTRMLTFKFYMPKKATELKHLQCLEEEL
KPLEEVLNLAQSKNFHLRPRDLISNINVIVLELKGSETTFMCEYADETATIVEFLNRWITFSQ
SIISTLT (SEQ ID NO: 2)
IL2-wt3
APASSSTKKTQLQLEHLLLDLQMILNGINNYKNPKLTRMLTFKFYMPKKATELKHLQCLEEEL
KPLEEVLNLAQSKNFHLRPRDLISNINVIVLELKGSETTFMCEYADETATIVEFLNRWITFSQ
SIISTLT (SEQ ID NO: 3)
IL2-G12 (de134-35PK)
APASSSTKKTQLQLEHLLLDLQMILNGINNYKNLTRMLTFKFYMPKKATELKHLQCLEEELKP
LEEVLNLAQSKNFHLRPRDLISNINVIVLELKGSETTFMCEYADETATIVEFLNRWITFSQSI
ISTLT (SEQ ID NO: 4)
IL2-G13 (de134-35PK, F42A)
APASSSTKKTQLQLEHLLLDLQMILNGINNYKNLTRMLTAKFYMPKKATELKHLQCLEEELKP
LEEVLNLAQSKNFHLRPRDLISNINVIVLELKGSETTFMCEYADETATIVEFLNRWITFSQSI
ISTLT (SEQ ID NO: 5)
IL2-D22 (del35K, Y45R)
APASSSTKKTQLQLEHLLLDLQMILNGINNYKNPLTRMLTFKFRMPKKATELKHLQCLEEELK
PLEEVLNLAQSKNFHLRPRDLISNINVIVLELKGSETTFMCEYADETATIVEFLNRWITFSQS
IISTLT (SEQ ID NO: 6)
IL2-D37 (de135K, F42A, Y45R)
APASSSTKKTQLQLEHLLLDLQMILNGINNYKNPLTRMLTAKFRMPKKATELKHLQCLEEELK
PLEEVLNLAQSKNFHLRPRDLISNINVIVLELKGSETTFMCEYADETATIVEFLNRWITFSQS
IISTLT (SEQ ID NO: 7)
Fc
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPG (SEQ ID NO: 8)
Linker
GGGGSGGGGSGGGGS (SEQ ID NO: 9)
WT: Fc-linker-IL2-wt3
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGGGGGSGGGGSGGGGSAPASSSTKKTQ
LQLEHLLLDLQMILNGINNYKNPKLTRMLTFKFYMPKKATELKHLQCLEEELKPLEEVLNLAQ
SKNFHLRPRDLISNINVIVLELKGSETTFMCEYADETATIVEFLNRWITFSQSIISTLT
(SEQ ID NO: 10)
G12: Fc-linker-IL2-G12
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGGGGGSGGGGSGGGGSAPASSSTKKTQ
LQLEHLLLDLQMILNGINNYKNLTRMLTFKFYMPKKATELKHLQCLEEELKPLEEVLNLAQSK
NFHLRPRDLISNINVIVLELKGSETTFMCEYADETATIVEFLNRWITFSQSIISTLT (SEQ
ID NO: 11)
G13: Fc-linker-IL2-G13
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGGGGGSGGGGSGGGGSAPASSSTKKTQ
LQLEHLLLDLQMILNGINNYKNLTRMLTAKFYMPKKATELKHLQCLEEELKPLEEVLNLAQSK
NFHLRPRDLISNINVIVLELKGSETTFMCEYADETATIVEFLNRWITFSQSIISTLT (SEQ
ID NO: 12)
D22: Fc-linker-IL2-D22
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGGGGGSGGGGSGGGGSAPASSSTKKTQ
LQLEHLLLDLQMILNGINNYKNPLTRMLTFKFRMPKKATELKHLQCLEEELKPLEEVLNLAQS
KNFHLRPRDLISNINVIVLELKGSETTFMCEYADETATIVEFLNRWITFSQSIISTLT (SEQ
ID NO: 13)
D37: Fc-linker-IL2-D37
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGGGGGSGGGGSGGGGSAPASSSTKKTQ
LQLEHLLLDLQMILNGINNYKNPLTRMLTAKFRMPKKATELKHLQCLEEELKPLEEVLNLAQS
KNFHLRPRDLISNINVIVLELKGSETTFMCEYADETATIVEFLNRWITFSQSIISTLT (SEQ
ID NO: 14)
PC: Fc-linker-IL2
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSK
LTVDKSRWQQGNVFSCSVMHEALHNHYTQKSLSLSPGGGGGSGGGGSGGGGSAPASSSTKKTQ
LQLEHLLLDLQMILNGINNYKNPKLTRMLTAKFAMPKKATELKHLQCLEEELKPLEEVLNGAQ
SLMFHRDLISNINVIVLELKGSETTFMCEYADETATIVEFLNRWITFAQSIISTLT (SEQ
ID NO: 15)
IL2-D37-1 (de135K, F42A, Y45R, E15Q)
APASSSTKKTQLQLQHLLLDLQMILNGINNYKNPLTRMLTAKFRMPKKATELKHLQCLEEELK
PLEEVLNLAQSKNFHLRPRDLISNINVIVLELKGSETTFMCEYADETATIVEFLNRWITFSQS
IISTLT (SEQ ID NO: 16)
IL2-D37-3 (de135K, F42A, Y45R, S87T)
APASSSTKKTQLQLEHLLLDLQMILNGINNYKNPLTRMLTAKFRMPKKATELKHLQCLEEELK
PLEEVLNLAQSKNFHLRPRDLITNINVIVLELKGSETTFMCEYADETATIVEFLNRWITFSQS
IISTLT (SEQ ID NO: 17)
IL2-D37-4 (de135K, F42A, Y45R, 88D)
APASSSTKKTQLQLEHLLLDLQMILNGINNYKNPLTRMLTAKFRMPKKATELKHLQCLEEELK
PLEEVLNLAQSKNFHLRPRDLISDINVIVLELKGSETTFMCEYADETATIVEFLNRWITFSQS
IISTLT (SEQ ID NO: 18)
IL2-D37-5 (de135K, F42A, Y45R, Q126T)
APASSSTKKTQLQLEHLLLDLQMILNGINNYKNPLTRMLTAKFRMPKKATELKHLQCLEEELK
PLEEVLNLAQSKNFHLRPRDLISNINVIVLELKGSETTFMCEYADETATIVEFLNRWITFSTS
IISTLT (SEQ ID NO: 19)
IL2-D37-6 (de135K, F42A, Y45R, D20L)
APASSSTKKTQLQLEHLLLLLQMILNGINNYKNPLTRMLTAKFRMPKKATELKHLQCLEEELK
PLEEVLNLAQSKNFHLRPRDLISNINVIVLELKGSETTFMCEYADETATIVEFLNRWITFSQS
IISTLT (SEQ ID NO: 20)
anti-PD-1 VH-CDR1
NYYMY (SEQ ID NO: 21)
anti-PD-1 VH-CDR2
GINPSNGGTNFNEKFKN (SEQ ID NO: 22)
anti-PD-1 VH-CDR3
RDYRFDMGFDY (SEQ ID NO: 23)
anti-PD-1 VL-CDR1
RASKGVSTSGYSYLH (SEQ ID NO: 24)
anti-PD-1 VL-CDR2
LASYLES (SEQ ID NO: 25)
anti-PD-1 VL-CDR3
QHSRDLPLT (SEQ ID NO: 26)
anti-PD-1 VH
QVQLVQSGVEVKKPGASVKVSCKASGYTFTNYYMYWVRQAPGQGLEWMGGINPSNGGTNFNEK
FKNRVTLTTDSSTTTAYMELKSLQFDDTAVYYCARRDYRFDMGFDYWGQGTTVTVSS (SEQ
ID NO: 27)
anti-PD-1 VL
EIVLTQSPATLSLSPGERATLSCRASKGVSTSGYSYLHWYQQKPGQAPRLLIYLASYLESGVP
ARFSGSGSGTDFTLTISSLEPEDFAVYYCQHSRDLPLTFGGGTKVEIK (SEQ ID NO:
28)
Heavy chain of half-antibody 309 (constnat region with
hole mutation, L234A/L235A/P329A, T366S/L368A/Y407V)
QVQLVQSGVEVKKPGASVKVSCKASGYTFTNYYMYWVRQAPGQGLEWMGGINPSNGGTNFNEK
FKNRVTLTTDSSTTTAYMELKSLQFDDTAVYYCARRDYRFDMGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLSCAVKGFYPS
DIAVEWESNGQPENNYKTTPPVLDSDGSFFLVSKLTVDKSRWQQGNVFSCSVMHEALHNHYTQ
KSLSLSP (SEQ ID NO: 29)
Light chain of half-antibody 309 and 309-0-309-6
EIVLTQSPATLSLSPGERATLSCRASKGVSTSGYSYLHWYQQKPGQAPRLLIYLASYLESGVP
ARFSGSGSGTDFTLTISSLEPEDFAVYYCQHSRDLPLTFGGGTKVEIKRTVAAPSVFIFPPSD
EQLKSGTASVVCLLNNFYPREAKVQWKVDNALQSGNSQESVTEQDSKDSTYSLSSTLTLSKAD
YEKHKVYACEVTHQGLSSPVTKSFNRGEC (SEQ ID NO: 30)
Long chain of half-antibody 309-0 (constant region with
knob mutation, L234A/L235A/P329A, T366W)
QVQLVQSGVEVKKPGASVKVSCKASGYTFTNYYMYWVRQAPGQGLEWMGGINPSNGGTNFNEK
FKNRVTLTTDSSTTTAYMELKSLQFDDTAVYYCARRDYRFDMGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLWCLVKGFYPS
DIAVEWESNGQPENNYKTTPPVLDSDGSFFLYSKLTVDKSRWQQGNVFSCSVMHEALHNHYTQ
KSLSLSPGGGGGSGGGGSGGGGSAPASSSTKKTQLQLEHLLLDLQMILNGINNYKNPLTRMLT
AKFRMPKKATELKHLQCLEEELKPLEEVLNLAQSKNFHLRPRDLISNINVIVLELKGSETTFM
CEYADETATIVEFLNRWITFSQSIISTLT (SEQ ID NO: 31)
Long chain of half-antibody 309-1 (constant region with
knob mutation, L234A/L235A/P329A, T366W)
QVQLVQSGVEVKKPGASVKVSCKASGYTFTNYYMYWVRQAPGQGLEWMGGINPSNGGTNFNEK
FKNRVTLTTDSSTTTAYMELKSLQFDDTAVYYCARRDYRFDMGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLWCLVKGFYPS
DIAVEWESNGQPENNYKTTPPVLDSDGSFFLYSKLTVDKSRWQQGNVFSCSVMHEALHNHYTQ
KSLSLSPGGGGGSGGGGSGGGGSAPASSSTKKTQLQLQHLLLDLQMILNGINNYKNPLTRMLT
AKFRMPKKATELKHLQCLEEELKPLEEVLNLAQSKNFHLRPRDLISNINVIVLELKGSETTFM
CEYADETATIVEFLNRWITFSQSIISTLT (SEQ ID NO: 32)
Long chain of half-antibody 309-3 (constant region with
knob mutation, L234A/L235A/P329A, T366W)
QVQLVQSGVEVKKPGASVKVSCKASGYTFTNYYMYWVRQAPGQGLEWMGGINPSNGGTNFNEK
FKNRVTLTTDSSTTTAYMELKSLQFDDTAVYYCARRDYRFDMGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLWCLVKGFYPS
DIAVEWESNGQPENNYKTTPPVLDSDGSFFLYSKLTVDKSRWQQGNVFSCSVMHEALHNHYTQ
KSLSLSPGGGGGSGGGGSGGGGSAPASSSTKKTQLQLEHLLLDLQMILNGINNYKNPLTRMLT
AKFRMPKKATELKHLQCLEEELKPLEEVLNLAQSKNFHLRPRDLITNINVIVLELKGSETTFM
CEYADETATIVEFLNRWITFSQSIISTLT (SEQ ID NO: 33)
Long chain of half-antibody 309-4 (constant region with
knob mutation, L234A/L235A/P329A, T366W)
QVQLVQSGVEVKKPGASVKVSCKASGYTFTNYYMYWVRQAPGQGLEWMGGINPSNGGTNFNEK
FKNRVTLTTDSSTTTAYMELKSLQFDDTAVYYCARRDYRFDMGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLWCLVKGFYPS
DIAVEWESNGQPENNYKTTPPVLDSDGSFFLYSKLTVDKSRWQQGNVFSCSVMHEALHNHYTQ
KSLSLSPGGGGGSGGGGSGGGGSAPASSSTKKTQLQLEHLLLDLQMILNGINNYKNPLTRMLT
AKFRMPKKATELKHLQCLEEELKPLEEVLNLAQSKNFHLRPRDLISDINVIVLELKGSETTFM
CEYADETATIVEFLNRWITFSQSIISTLT (SEQ ID NO: 34)
Long chain of half-antibody 309-5 (constant region with
knob mutation, L234A/L235A/P329A, T366W)
QVQLVQSGVEVKKPGASVKVSCKASGYTFTNYYMYWVRQAPGQGLEWMGGINPSNGGTNFNEK
FKNRVTLTTDSSTTTAYMELKSLQFDDTAVYYCARRDYRFDMGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLWCLVKGFYPS
DIAVEWESNGQPENNYKTTPPVLDSDGSFFLYSKLTVDKSRWQQGNVFSCSVMHEALHNHYTQ
KSLSLSPGGGGGSGGGGSGGGGSAPASSSTKKTQLQLEHLLLDLQMILNGINNYKNPLTRMLT
AKFRMPKKATELKHLQCLEEELKPLEEVLNLAQSKNFHLRPRDLISNINVIVLELKGSETTFM
CEYADETATIVEFLNRWITESTSIISTLT (SEQ ID NO: 35)
Long chain of half-antibody 309-6 (constant region with
knob mutation, L234A/L235A/P329A, T366W)
QVQLVQSGVEVKKPGASVKVSCKASGYTFTNYYMYWVRQAPGQGLEWMGGINPSNGGTNFNEK
FKNRVTLTTDSSTTTAYMELKSLQFDDTAVYYCARRDYRFDMGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPEAAGGPSVFLFPPKPKD
TLMISRTPEVTCVVVDVSHEDPEVKFNWYVDGVEVHNAKTKPREEQYNSTYRVVSVLTVLHQD
WLNGKEYKCKVSNKALAAPIEKTISKAKGQPREPQVYTLPPSRDELTKNQVSLWCLVKGFYPS
DIAVEWESNGQPENNYKTTPPVLDSDGSFFLYSKLTVDKSRWQQGNVFSCSVMHEALHNHYTQ
KSLSLSPGGGGGSGGGGSGGGGSAPASSSTKKTQLQLEHLLLLLQMILNGINNYKNPLTRMLT
AKFRMPKKATELKHLQCLEEELKPLEEVLNLAQSKNFHLRPRDLISNINVIVLELKGSETTFM
CEYADETATIVEFLNRWITFSQSIISTLT (SEQ ID NO: 36)
Anti-LAG-3 (MIL98) VH-CDR1
DYEMH (SEQ ID NO: 37)
Anti-LAG-3 (MIL98) VH-CDR2
AIDPETGGIVYNQRFKG (SEQ ID NO: 38)
Anti-LAG-3 (MIL98) VH-CDR3
TGWND (SEQ ID NO: 39)
Anti-LAG-3 (MIL98) VL-CDR1
KASQDINSYLS (SEQ ID NO: 40)
Anti-LAG-3 (MIL98) VL-CDR2
RANRLLD (SEQ ID NO: 41)
Anti-LAG-3 (MIL98) VL-CDR3
QQYDEFPFT (SEQ ID NO: 42)
Anti-LAG-3 VH
QVQLVQSGAEVKKPGASVKVSCKVSGYTFTDYEMHWVRQAPGKGLEWMGAIDPETGGIVYNQR
FKGRVTLTADTSTDTAYMELSSLRSEDTAVYYCATTGWNDWGQGTTVTVSS (SEQ ID NO:
43)
Anti-LAG-3 VL
DIQMTQSPSSLSASVGDRVTITCKASQDINSYLSWYQQKPGKAPKLLIYRANRLLDGVPSRFS
GSGSGQDYTLTISSLQPEDFATYYCQQYDEFPFTFGQGTKLEIK (SEQ ID NO: 44)
Long chain of anti-LAG-3 half-antibody (constant region
with hole mutation, L234A/L235A/P329A, T366S/L368A/Y407V)
QVQLVQSGAEVKKPGASVKVSCKVSGYTFTDYEMHWVRQAPGKGLEWMGAIDPETGGIVYNQR
FKGRVTLTADTSTDTAYMELSSLRSEDTAVYYCATTGWNDWGQGTTVTVSSASTKGPSVFPLA
PSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPEAAGGPSVFLFPPKPKDTLMISR
TPEVTCVVVDVSHEDPEVKFNWYVDGVEVHNAKTKPREEQYNSTYRVVSVLTVLHQDWLNGKE
YKCKVSNKALAAPIEKTISKAKGQPREPQVCTLPPSRDELTKNQVSLSCAVKGFYPSDIAVEW
ESNGQPENNYKTTPPVLDSDGSFFLVSKLTVDKSRWQQGNVFSCSVMHEALHNHYTQKSLSLS
PGK (SEQ ID NO: 45)
Light chain of anti-LAG-3 half-antibody
DIQMTQSPSSLSASVGDRVTITCKASQDINSYLSWYQQKPGKAPKLLIYRANRLLDGVPSRFS
GSGSGQDYTLTISSLQPEDFATYYCQQYDEFPFTFGQGTKLEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC (SEQ ID NO: 46)
hPD-1
MQIPQAPWPVVWAVLQLGWRPGWFLDSPDRPWNPPTFSPALLVVTEGDNATFTCSFSNTSESF
VLNWYRMSPSNQTDKLAAFPEDRSQPGQDCRFRVTQLPNGRDFHMSVVRARRNDSGTYLCGAI
SLAPKAQIKESLRAELRVTERRAEVPTAHPSPSPRPAGQFQTLVVGVVGGLLGSLVLLVWVLA
VICSRAARGTIGARRTGQPLKEDPSAVPVFSVDYGELDFQWREKTPEPPVPCVPEQTEYATIV
FPSGMGTSSPARRGSADGPRSAQPLRPEDGHCSWPL (SEQ ID NO: 47)

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An interleukin-2 (IL-2) variant, comprising
an amino acid residue deletion, and a Phe to Ala substitution at sites corresponding to Position 35 and 42 of SEQ ID NO: 1, respectively;
an amino acid residue deletion, and a Tyr to Arg substitution at sites corresponding to Position 35 and 45 of SEQ ID NO: 1, respectively; or
two residue deletions at sites corresponding to Position 34 and 35 of SEQ ID NO: 1, respectively.

2. The IL-2 variant of claim 1, comprising
an amino acid residue deletion, a Phe to Ala substitution, and a Tyr to Arg substitution at sites corresponding to Position 35, 42 and 45 of SEQ ID NO: 1, respectively;
an amino acid residue deletion, and a Tyr to Arg substitution at sites corresponding to Position 35 and 45 of SEQ ID NO: 1, respectively; or
two residue deletions at sites corresponding to Position 34 and 35 of SEQ ID NO: 1, and a Phe to Ala substitution at site corresponding to Position 42 of SEQ ID NO: 1.

3. The IL-2 variant of claim 2, further comprising one or more selected from the group consisting of an Asp to leu substitution at a site corresponding to Position 20 of SEQ ID NO: 1, an Asn to Asp substitution at a site corresponding to Position 88 of SEQ ID NO: 1, and a Gln to Thr substitution at a site corresponding to Position 126 of SEQ ID NO: 1.

4. The IL-2 variant of claim 3, further comprising a Thr to Ala substitution, a Thr to Gly substitution, a Thr to Gln substitution, a Thr to Glu substitution, a Thr to Asn substitution, a Thr to Asp substitution, a Thr to Arg substitution, a Thr to Lys substitution, or a Thr to Pro substitution at a site corresponding to Position 3 of SEQ ID NO: 1, and/or
a Cys to Ser substitution, or a Cys to Ala substitution at a site corresponding to Position 125 of SEQ ID NO: 1.

5. The IL-2 variant of claim 4, comprising the amino acid sequence of SEQ ID NOs: 18, 19 or 20.

6. A recombinant protein, comprising:
i) the IL-2 variant of claim 1, and
ii) an antigen binding domain targeting a non-IL-2 molecule,
wherein the IL-2 variant is linked to the antigen binding domain.

7. The recombinant protein of claim 6,
wherein the antigen binding domain targeting a non-IL-2 molecule is an anti-PD-1 antibody or an antigen binding portion thereof, comprising an anti-PD-1 heavy chain variable region and an anti-PD-1 light chain variable region,
wherein the anti-PD-1 heavy chain variable region comprises a heavy chain variable region (VH)-CDR1, a VH-CDR2, and a VH-CDR3, the anti-PD-1 light chain variable region comprises a light chain variable region (VL)-CDR1, a VL-CDR2 and a VL-CDR3, wherein the VH-CDR1, the VH-CDR2, the VH-CDR3, the VL-CDR1, the VL-CDR2 and the VL-CDR3 comprise the amino acid sequences of SEQ ID NOs: 21-26, respectively.

8. The recombinant protein of claim 7, comprising:
i) a first polypeptide, comprising the anti-PD-1 heavy chain variable region, a heavy chain constant region, and the IL-2 variant,
ii) a second polypeptide, comprising the anti-PD-1 light chain variable region and a light chain constant region,
iii) a third polypeptide, comprising the anti-PD-1 heavy chain variable region, and a heavy chain constant region, and
iv) a fourth polypeptide, comprising the anti-PD-1 light chain variable region and a light chain constant region,
wherein the anti-PD-1 heavy chain variable region in the first polypeptide and the anti-PD-1 light chain variable region in the second polypeptide form the antigen binding domain targeting a non-IL-2 molecule, the anti-PD-1 heavy chain variable region in the third polypeptide and the anti-PD-1 light chain variable region in the fourth polypeptide form the antigen binding domain targeting a non-IL-2 molecule, the heavy chain constant region in the first polypeptide and the heavy chain constant region in the third polypeptide are associated together.

9. The recombinant protein of claim 7, further comprising an anti-LAG-3 antibody or an antigen binding portion thereof which comprises an anti-LAG-3 heavy chain variable region and an anti-LAG-3 light chain variable region, wherein the anti-LAG-3 heavy chain variable region comprises a heavy chain variable region (VH)-CDR1, a VH-CDR2, and a VH-CDR3, the anti-LAG-3 light chain variable region comprises a VL CDR4 light chain variable region (VH)-CDR1, a VL-CDR2 and a VL-CDR3, wherein the VH-CDR1, the VH-CDR2, the VH-CDR3, the VL-CDR1, the VL-CDR2 and the VL-CDR3 comprise the amino acid sequences of SEQ ID NOs: 37-42, respectively.

10. The recombinant protein of claim 9, comprising:
i) a first polypeptide, comprising the anti-PD-1 heavy chain variable region, a heavy chain constant region, and the IL-2 variant,
ii) a second polypeptide, comprising the anti-PD-1 light chain variable region and a light chain constant region,
iii) a third polypeptide, comprising the anti-LAG-3 heavy chain variable region, and a heavy chain constant region, and
iv) a fourth polypeptide, comprising the anti-LAG-3 light chain variable region and a light chain constant region,
wherein the anti-PD-1 heavy chain variable region in the first polypeptide and the anti-PD-1 light chain variable region in the second polypeptide form the antigen binding domain targeting a non-IL-2 molecule, the anti-LAG-3 heavy chain variable region in the third polypeptide and the anti-LAG-3 light chain variable region in the fourth polypeptide form the anti-LAG-3 antibody or the antigen binding portion thereof, the heavy chain constant region in the first polypeptide and the heavy chain constant region in the third polypeptide are associated together.

11. The recombinant protein of claim 10, wherein,
the first polypeptide comprises, from N terminus to C terminus, the anti-PD-1 heavy chain variable region, the heavy chain constant region, and the IL-2 variant,
the second polypeptide comprises, from N terminus to C terminus, the anti-PD-1 light chain variable region and the light chain constant region,
the third polypeptide comprises, from N terminus to C terminus, the anti-PD-1 heavy chain variable region, and the heavy chain constant region,
the fourth polypeptide comprises, from N terminus to C terminus, the anti-PD-1 light chain variable region and the light chain constant region.

12. The recombinant protein of claim 8, wherein the heavy chain constant regions in the first polypeptide and the third polypeptide are each IgG1 heavy chain constant region comprising L234A/L235A/P329A mutations, or human IgG4 heavy chain constant region.

13. The recombinant protein of claim 12, wherein the heavy chain constant region in the first polypeptide is IgG heavy chain constant region comprising L234A/L235A/P329A/T366W mutations, and the heavy chain constant region in the third polypeptide is IgG1 heavy chain constant region comprising L234A/L235A/P329A/T366S/L368A/Y407V mutations; or
the heavy chain constant region in the first polypeptide is IgG1 heavy chain constant region comprising L234A/L235A/P329A/T366S/L368A/Y407V mutations, and the heavy chain constant region in the third polypeptide is IgG heavy chain constant region comprising L234A/L235A/P329A/T366W mutations.

14. The recombinant protein of claim 13, wherein the first, second, third and fourth polypeptides respectively comprise the amino acid sequences of i) SEQ ID NOs: 34, 30, 29 and 30; ii) SEQ ID NOs: 35, 30, 29 and 30; or iii) SEQ ID NOs: 36, 30, 29 and 30.

15. An isolated nucleic acid molecule, encoding the IL-2 variant of claim 1.

16-17. (canceled)

18. A composition, comprising the recombinant protein of claim 6.

19. (canceled)

20. The recombinant protein of claim 10, wherein,
the first polypeptide comprises, from N terminus to C terminus, the anti-PD-1 heavy chain variable region, the heavy chain constant region, and the IL-2 variant,
the second polypeptide comprises, from N terminus to C terminus, the anti-PD-1 light chain variable region and the light chain constant region,
the third polypeptide comprises, from N terminus to C terminus, the anti-LAG-3 heavy chain variable region, and the heavy chain constant region,
the fourth polypeptide comprises, from N terminus to C terminus, the anti-LAG-3 light chain variable region and the light chain constant region.

21. The recombinant protein of claim 20, wherein the heavy chain constant region in the first polypeptide is IgG heavy chain constant region comprising L234A/L235A/P329A/T366W mutations, and the heavy chain constant region in the third polypeptide is IgG1 heavy chain constant region comprising L234A/L235A/P329A/T366S/L368A/Y407V mutations; or
the heavy chain constant region in the first polypeptide is IgG1 heavy chain constant region comprising L234A/L235A/P329A/T366S/L368A/Y407V mutations, and the heavy chain constant region in the third polypeptide is IgG heavy chain constant region comprising L234A/L235A/P329A/T366W mutations.

22. The recombinant protein of claim 21, wherein the first, second, third and fourth polypeptides respectively comprise the amino acid sequences of i) SEQ ID NOs: 31, 30, 45 and 46; ii) SEQ ID NOs: 34, 30, 45 and 46; iii) SEQ ID NOs: 35, 30, 45 and 46; or iv) SEQ ID NOs: 36, 30, 45 and 46.

23. The IL-2 variant of claim 2, comprising the amino acid sequence of SEQ ID NOs: 5, 6 or 7.


SEQUENCE LISTING (47 sequences)

SEQ ID NO: 1 - protein, 133 aa, Homo sapiens [sequence omitted]

SEQ ID NO: 2 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - protein, 131 aa, synthetic construct
APASSSTKKTQLQLEHLLLDLQMILNGINNYKNLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 131 aa, synthetic construct
APASSSTKKTQLQLEHLLLDLQMILNGINNYKNLTRMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 6 - protein, 132 aa, synthetic construct
APASSSTKKTQLQLEHLLLDLQMILNGINNYKNPLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 7 - protein, 132 aa, synthetic construct
APASSSTKKTQLQLEHLLLDLQMILNGINNYKNPLTRMLTAKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 8 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 15 aa, synthetic construct
GGGGSGGGGSGGGGS

SEQ ID NO: 10 - protein, 374 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 11 - protein, 372 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 12 - protein, 372 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNLTRMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 13 - protein, 373 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPLTRMLTFKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 14 - protein, 373 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPLTRMLTAKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 15 - protein, 371 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSLMFHRDLISNINVIVLELKGSETTFMCEYADETATIVEFLNRWITFAQSIISTLT

SEQ ID NO: 16 - protein, 132 aa, synthetic construct
APASSSTKKTQLQLQHLLLDLQMILNGINNYKNPLTRMLTAKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 17 - protein, 132 aa, synthetic construct
APASSSTKKTQLQLEHLLLDLQMILNGINNYKNPLTRMLTAKFRMPKKATELKHLQCLEEELKPLEEVLNLAQSKNFHLRPRDLITNINVIVLELKGSETTFMCEYADETATIVEFLNRWITFSQSIISTLT

SEQ ID NO: 18 - protein, 132 aa, synthetic construct
APASSSTKKTQLQLEHLLLDLQMILNGINNYKNPLTRMLTAKFRMPKKATELKHLQCLEEELKPLEEVLNLAQSKNFHLRPRDLISDINVIVLELKGSETTFMCEYADETATIVEFLNRWITFSQSIISTLT

SEQ ID NO: 19 - protein, 132 aa, synthetic construct
APASSSTKKTQLQLEHLLLDLQMILNGINNYKNPLTRMLTAKFRMPKKATELKHLQCLEEELKPLEEVLNLAQSKNFHLRPRDLISNINVIVLELKGSETTFMCEYADETATIVEFLNRWITFSTSIISTLT

SEQ ID NO: 20 - protein, 132 aa, synthetic construct
APASSSTKKTQLQLEHLLLLLQMILNGINNYKNPLTRMLTAKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 21 - protein, 5 aa, synthetic construct
NYYMY

SEQ ID NO: 22 - protein, 17 aa, synthetic construct
GINPSNGGTNFNEKFKN

SEQ ID NO: 23 - protein, 11 aa, synthetic construct
RDYRFDMGFDY

SEQ ID NO: 24 - protein, 15 aa, synthetic construct
RASKGVSTSGYSYLH

SEQ ID NO: 25 - protein, 7 aa, synthetic construct
LASYLES

SEQ ID NO: 26 - protein, 9 aa, synthetic construct
QHSRDLPLT

SEQ ID NO: 27 - protein, 120 aa, synthetic construct [sequence omitted]

SEQ ID NO: 28 - protein, 111 aa, synthetic construct [sequence omitted]

SEQ ID NO: 29 - protein, 448 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 30 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 596 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPLTRMLTAKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 32 - protein, 596 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLQHLLLDLQMILNGINNYKNPLTRMLTAKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 33 - protein, 596 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPLTRMLTAKFRMPKKATELKHLQCLEEELKPLEEVLNLAQSKNFHLRPRDLITNINVIVLELKGSETTFMCEYADETATIVEFLNRWITFSQSIISTLT

SEQ ID NO: 34 - protein, 596 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPLTRMLTAKFRMPKKATELKHLQCLEEELKPLEEVLNLAQSKNFHLRPRDLISDINVIVLELKGSETTFMCEYADETATIVEFLNRWITFSQSIISTLT

SEQ ID NO: 35 - protein, 596 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPLTRMLTAKFRMPKKATELKHLQCLEEELKPLEEVLNLAQSKNFHLRPRDLISNINVIVLELKGSETTFMCEYADETATIVEFLNRWITFSTSIISTLT

SEQ ID NO: 36 - protein, 596 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLLLQMILNGINNYKNPLTRMLTAKFRMPKKATELKHLQCLEEELKPLEEVLNLAQSKNFHLRPRDLISNINVIVLELKGSETTFMCEYADETATIVEFLNRWITFSQSIISTLT

SEQ ID NO: 37 - protein, 5 aa, synthetic construct
DYEMH

SEQ ID NO: 38 - protein, 17 aa, synthetic construct
AIDPETGGIVYNQRFKG

SEQ ID NO: 39 - protein, 5 aa, synthetic construct
TGWND

SEQ ID NO: 40 - protein, 11 aa, synthetic construct
KASQDINSYLS

SEQ ID NO: 41 - protein, 7 aa, synthetic construct
RANRLLD

SEQ ID NO: 42 - protein, 9 aa, synthetic construct
QQYDEFPFT

SEQ ID NO: 43 - protein, 114 aa, synthetic construct
QVQLVQSGAEVKKPGASVKVSCKVSGYTFTDYEMHWVRQAPGKGLEWMGAIDPETGGIVYNQRFKGRVTLTADTSTDTAYMELSSLRSEDTAVYYCATTGWNDWGQGTTVTVSS

SEQ ID NO: 44 - protein, 107 aa, synthetic construct
DIQMTQSPSSLSASVGDRVTITCKASQDINSYLSWYQQKPGKAPKLLIYRANRLLDGVPSRFSGSGSGQDYTLTISSLQPEDFATYYCQQYDEFPFTFGQGTKLEIK

SEQ ID NO: 45 - protein, 444 aa, synthetic construct
QVQLVQSGAEVKKPGASVKVSCKVSGYTFTDYEMHWVRQAPGKGLEWMGAIDPETGGIVYNQRFKGRVTLTADTSTDTAYMELSSLRSEDTAVYYCATTGWNDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 214 aa, synthetic construct
DIQMTQSPSSLSASVGDRVTITCKASQDINSYLSWYQQKPGKAPKLLIYRANRLLDGVPSRFSGSGSGQDYTLTISSLQPEDFATYYCQQYDEFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL